(19) United States
(12) Patent Application Publication
(10) Pub. No.: US 2016/0130328 A1
(43) Pub. Date: May 12, 2016

(54) Pathogen Binding Agents Conjugated to Radioisotopes and Uses in Imaging and Therapeutic Applications

(71) Applicants: EMORY UNIVERSITY, Atlanta, GA (US); GEORGIA TECH RESEARCH CORPORATION, Atlanta, GA (US)

(72) Inventors: Philip SANTANGELO, Winder, GA (US); Francois VILLINGER, Decatur, GA (US)

(21) Appl. No.: 14/899,284

(22) PCT Filed: Jun. 13, 2014
(86) PCT No.: PCT/US2014/042264

§ 371 Date: Dec. 17, 2015

Related U.S. Application Data
(60) Provisional application No. 61/835,840, filed on Jun. 17, 2013.

(51) Int. Cl. C07K 16/10; A61K 47/48; A61K 45/06; A61K 51/10

(52) U.S. Cl. CPC C07K 16/1063; A61K 51/1096; A61K 51/1006; A61K 47/4853; A61K 45/06; C07K 2317/56; C07K 2317/24; C07K 2317/565; C07K 2317/14; A61K 2039/505

(57) ABSTRACT

This disclosure relates to binding agents specific for the envelope protein of a virus, e.g., lentivirus, wherein the binding agent is conjugated to a molecule with a radioisotope or positron-emitting radionuclide. In certain embodiments, the disclosure relates to methods of imaging a virus or other pathogen within the body of a subject using binding agents disclosed herein. In certain embodiments, the disclosure relates to methods of treating or preventing a viral or other pathogenic infection by administering pharmaceutical composition containing radioactive binding agents disclosed herein.

### CROSS-REFERENCE TO RELATED APPLICATIONS

This application claims priority to U.S. Provisional Application No. 61/835,840 filed Jun. 17, 2013, hereby incorporated by reference in its entirety.

### STATEMENT REGARDING FEDERALLY FUNDED RESEARCH

This invention was made with government support under Grant R21 AI095129-01A1 awarded by the National Institutes of Health. The government has certain rights in the invention.

### BACKGROUND

Antiretroviral therapies have made considerable progress by providing HIV infected patients with drug cocktails able to lower viral loads to undetectable levels. However, these therapies are unable to eliminate the virus from the host eventually leading to a rapid re-emergence of viremia to pre-treatment levels when treatment is discontinued. Considerable efforts have been devoted to understand the seeding and long-term maintenance of HIV viral reservoirs, however, they are complex, and it is likely that residual replication is maintained in even well medicated hosts due to sanctuaries. Thus, there is a need to understand the spatial dynamics for virus resurgence, continuous replication, as well as the initial dissemination during acute infection in order to evaluate potential therapeutics and vaccines.

Tools available to monitor such viral processes are typically indirect or very invasive. Even when one uses a non-human primate model of AIDS, understanding the viral dynamics in real time is challenging and prohibitively expensive. Measuring the effectiveness of individual antiretroviral drugs, preventive and therapeutic vaccines, immunotherapies and other therapies would benefit from a more precise monitoring of spatial viral replication in vivo. Thus, there is a need to develop a sensitive, specific and non-invasive method for monitoring the dynamics and dissemination of HIV.

Sathekge et al. report positron emission tomography in patients suffering from HIV-1 infection. See Eur J Nucl Med Mol Imaging, 2009, 36(7):1176-84. See also Lucignani et al., Eur J Nucl Med Mol Imaging, 2009, 36(4): 640-7. These techniques are not targeted to the virus and are prone to a number of influences other than virus replication alone. Thus, there is a need for the direct assessment of a lentiviral infection in-vivo.

Leung reports ⁶⁴Cu-DOTA conjugated to an inhibitor of CXCR4 activity has been studied with positron emission tomography (PET). See Molecular Imaging and Contrast Agent Database (MICAD) available at http://www.ncbi.nlm.nih.gov/books/NBK84043/. Li et al. report monodispersed DOTA-PEG-conjugated anti-TAG-72 diabody has low kidney uptake and high tumor-to-blood ratios. See J Nucl Med, 2010, 51(7):1139-46. See also Veronese et al., BioDrugs, 2008. 22(5): 315-29.

References cited herein are not an admission of prior art.

### SUMMARY

This disclosure relates to binding agents specific for the envelope protein of a virus, e.g., lentivirus, wherein the binding agent is conjugated to a molecule with a radioisotope or positron-emitting radionuclide. In certain embodiments, the disclosure relates to methods of imaging a virus or other pathogen within the body of a subject using binding agents disclosed herein. In certain embodiments, the disclosure relates to methods of treating or preventing a viral or other pathogenic infection by administering pharmaceutical composition containing radioactive binding agents disclosed herein.

In certain embodiments, the disclosure relates to binding agents specific for pathogenic antigen, wherein the binding agent is conjugated to a molecule with a positron-emitting radionuclide. In certain embodiments, the pathogenic antigen is a virus particle surface antigen expressed on virions or infected cells. In certain embodiments, the binding agent is an antibody with an epitope to a gp120 protein of a lentivirus, e.g., the V3 loop of a gp120 protein of a lentivirus such as a simian immunodeficiency virus or human immunodeficiency virus.

In certain embodiments, the binding agent is the monoclonal antibody. In certain embodiments, the binding agent is a humanized antibody or human chimera.

In certain embodiments, the binding agent is a human chimera comprises a polypeptide sequence selected from a) a variable domain of the light chain from an antibody conjugated to a human immunoglobulin; b) a variable domain of the heavy chain from an antibody to a human immunoglobulin; or c) a variable domain of the light chain and heavy chain from an antibody conjugated to a human immunoglobulin.

In certain embodiments, the binding agent is humanized antibody comprises polypeptide sequences of complementarity determining region one (CDR-1), CDR-2, and CDR-3 on the light (V_{L}) chain of an antibody and polypeptide sequences of CDR-1, CDR-2, and CDR-3 heavy (V_{H}) chains of an antibody.

In certain embodiment, the binding agent is the antibody is selected from CD4BS, CH103, PG V04, PGT-127, PGT-128, PGT-130, PGT-131, CH01, CH02, CH03, and CH04, 2909, VRC01, VRC02, VRC03, HJ16, HGN194, HK20, PG9, PG16, 22A, 171C2, 71B7, 36D5, 31C7, 8H1, 189D5, 77D6, 3E9, 4B11, 5B11, 7D3, 8C7, 11F2, 17A11, 2G12, b12, b13, m18, F105, and 447-52D.

In certain embodiments, the specific binding agent comprises 1,4,7,10-tetraazacyclododecane as a chelating moiety. In certain embodiments, the specific binding agent is an antibody conjugated to a hydrophilic polymer such as polyethylene glycol.

In certain embodiments, the disclosure relates to methods of imaging a lentiviral infection comprising, a) administering a tracer composition comprising a specific binding agent of disclosed herein to a subject; b) detecting pairs of gamma rays emitted by the positron-emitting radionuclide; and c) generating an image indicating a location of the positron-emitting radionuclide within an area of the subject.

In certain embodiments, the disclosure relates to methods of treating or preventing a pathogenic infection such as a viral or lentiviral infection comprising administering an effective amount of a specific binding agent for a lentivirus envelope protein or other pathogenic antigen, wherein the binding agent is conjugated to a molecule with a radioisotope or positron-emitting radionuclide or, to a subject in need thereof. In certain embodiments, the subject is human. In certain embodiments, the specific binding agent is administered in combination with another antiviral agent such as abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, elvitegravir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, MK-2048, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate (TAF), tipranavir, trifluridine, trizivir, tromantadine, truvada, valaciclovir, valganciclovir, vicriviroc, vidarabine, viramidine, zalcitabine, zanamivir, or zidovudine, and combinations thereof.

### BRIEF DESCRIPTION OF THE FIGURES

FIG. 1 shows PET/CT results from uninfected control and chronically SW infected, viremic rhesus macaques. a,b,c,d. Frontal, sagittal and axial views are presented, and magnified views of regions of the frontal and sagittal sections for 2 viremic monkeys RFR11 and RID9 as well as for a representative uninfected monkey RHG7 imaged with the 7D3 or control IgG probe. Axial sections are identified within the sagittal view, denoted by a yellow line. RFR11 and RID9 demonstrated PET signal within the GI tract, axillary and inguinal lymph nodes, genital tract and lungs. Data obtained by scanning of the uninfected monkey RHG7 shows that the background signal detected was significantly lower than in the infected animals, even within the liver, heart, and kidneys, sites for which background was expected. e,f,g. IHC against SIV gag for macaques RFR11 (e), RID9 (f) and uninfected control tissue (g): Infected mononuclear cells (arrows) were detected in the ileum, jejunum, and colon, as well as within lymph nodes and the spleen, confirming the PET results. h. Quantification of the PET data. The maximum standard uptake value (SUVmax) within each organ was measured, and was then compared directly with the qRT-PCR results. i. Rectal biopsy results from 2 chronically infected and 2 non-infected animals indicate substantial uptake of probe within the infected but not in uninfected animals. j. Comparison of SUVmax results in viremic vs uninfected monkeys; a repeated measures ANOVA analysis confirmed that the animal conditions were statistically different. k. Measurement of the SUV ratio at two time points following probe injection. The ratios for the infected monkeys, were consistently greater than for the aviremic controls, typically greater than 0.6, with the GI tract ratio consistently >1.0, indicating continued specific uptake of the contrast agent.

FIG. 2 shows PET/CT results from chronically infected macaques, before and at five weeks of ART treatment. PET/CT images of SIV chronically infected macaques prior to and at 4 weeks of ART. a. Standard uptake value (SUV) maps of GI tract, lymph nodes, genital tract, spleen and small bowel, demonstrating decreased probe uptake after 4 weeks of ART. b. SUVmax values before and after 4 weeks of ART, compared with background uptake in non-infected animals. c. qRT-PCR verification of residual virus compared with SUVmax PET data.

FIG. 3 shows PET/CT results from SIV infected, elite controllers (EC). Frontal, sagittal and axial views are presented, as well as magnified views of the frontal and sagittal sections (marked by a colored box and associated image denoted by the same color outline). The axial section presented is identified within the sagittal view, denoted by a yellow line. SUV scale bars are presented for each image. a. PET/CT single plane cross-sections from three SIV infected, EC macaques, 36 hrs post injection of the labeled antibody. Macaques RBQ10, RUN10, and RMP10 (Extended data FIG. 7), were infected for over 6 years, and displayed plasma viral loads less than 60 copies of viral RNA/m1 for the last 5 years. Uptake was apparent within lungs, NALT, genital tract and the GI tract. GI signal was less diffuse than in chronically infected animals, restricted to foci within mesenteric lymph nodes (see blue boxes). b. SUVmax quantification results from the PET/CT imaging, comparing viremic and EC monkeys with uninfected controls. See Extended data for a description of the statistical analysis of this data. c. IHC results against the SIVmac239 gag for macaques RBQ10 and RUN10, respectively. Infected mononuclear cells were detected in tissue sections of biopsies of the rectum and epididymis.

FIG. 4 shows a comparison of viremic to elite controller macaques. a. Comparison of SUVmean and SUV voxel fraction (fraction of total volume of GI tract) within the GI tract of chronic SIV+ and EC macaques. The voxel fractions that contained SUVs above 1 and less than 3.3 (excludes interfering signals) were included in this graph. b. Haralick texture function measurements of angular second moment and contrast for ROIs (depicted by red boxes). The angular second moment is a measure of homogeneity, while the contrast metric represents the local variations within an image or ROI. These metrics are inversely proportional to each other, and therefore ideal to compare the distributions of signal within the GI tract of chronically infected animals and elite controllers. c,d. Representative axial cross sections of two chronically infected macaques (RFR11 and RID9) and two controllers macaques (RMP10 and RBQ10), respectively. The uptake in the chronically viremic infected animals (white arrows) tend to follow the length of the GI organs, while in the controllers (white arrows), the signal localizes to specific foci, some of which are mesenteric lymph nodes (from CT), while in other cases they correspond to specific regions of the small intestines or colon. The red arrows indicate uptake within the liver.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, a “conjugate” refers to any molecule that contains covalently linked identified moieties produced by synthetic or recombinant techniques. In some instances the moieties are metal binding ligands to provide a metal radioisotope bound to multi-dentate ligands further conjugated to a specific binding agent, e.g., an antibody. The moieties are typically substituted and coupled together and separated by linking groups containing amides, esters, peptides, hydrocarbons, glycols, polyethylene glycols, or other polymeric groups and the like.

As used herein, the term “combination with” when used to describe administration with an additional treatment means that the agent may be administered prior to, together with, or after the additional treatment, or a combination thereof.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the condition or disease is eradicated. Rather, embodiments, of the present disclosure also contemplate treatment that merely reduces symptoms, and/or delays conditions or disease progression.

The term “a pathogenic specific binding agent” refers to a molecule, preferably a proteinaceous molecule that binds pathogenic antigen or in certain instances antigen exposed on the particle or cell of pathogen with a greater affinity than other pathogen produced proteins or domains, e.g., viral envelope proteins, glycoproteins, saccharides, polysaccharides, and gp120. Typically the specific binding agent is an antibody, such as a polyclonal antibody, a monoclonal antibody (mAb), a chimeric antibody, a CDR-grafted antibody, a multi-specific antibody, a bi-specific antibody, a catalytic antibody, a humanized antibody, a human antibody, an anti-idiotypic (anti-Id) antibody, and antibodies that can be labeled in soluble or bound form, as well as antigen-binding fragments, variants or derivatives thereof, either alone or in combination with other amino acid sequences, provided by known techniques.

The term “polyclonal antibody” refers to a heterogeneous mixture of antibodies that recognize and bind to different epitopes on the same antigen. Polyclonal antibodies may be obtained from crude serum preparations or may be purified using, for example, antigen affinity chromatography, or Protein A/Protein G affinity chromatography.

The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same epitope on the antigen. The term “monoclonal” is not limited to any particular method for making the antibody, nor is the term limited to antibodies produced in a particular species, e.g., mouse, rat, etc.

The term “chimeric antibodies” refers to antibodies in which a portion of the heavy and/or light chain is identical with or homologous to a corresponding sequence in an antibody derived from a particular species or belonging to a particular antibody class or subclass, while the remainder of the chain(s) is/are identical with or homologous to a corresponding sequence in antibodies derived from another species or belonging to another antibody class or subclass. Also included are antigen-binding fragments of such antibodies that exhibit the desired activity (e.g, the ability to specifically bind gp120). See, U.S. Pat. No. 4,816,567 and Morrison et al., Proc Natl Acad Sci (USA), 81:6851-6855 [1985].

The term “CDR grafted antibody” refers to an antibody in which the CDR from one antibody of a particular species or isotype is recombinantly inserted into the framework of another antibody of the same or different species or isotype.

The term “multi-specific antibody” refers to an antibody having variable regions that recognize more than one epitope on one or more antigens. A subclass of this type of antibody is a “bi-specific antibody” which recognizes two distinct epitopes on the same or different antigens.

The term “humanized antibody” refers to a specific type of CDR-grafted antibody in which the antibody framework region is derived from a human but each CDR is replaced with that derived from another species, such as a murine CDR. The term “CDR” is defined infra.

The term “fully human” antibody refers to an antibody in which both the CDR and the framework are derived from one or more human DNA molecules.

The term “variants,” as used herein, include those polypeptides wherein amino acid residues are inserted into, deleted from and/or substituted into the naturally occurring (or at least a known) amino acid sequence for the binding agent. Variants of the disclosure include fusion proteins as described below.

“Specifically binds” refers to the ability of a specific binding agent (such as an antibody or fragment thereof) of the present disclosure to recognize and bind mature, full-length or partial-length target polypeptide (e.g., gp120), or an ortholog thereof, such that its affinity (as determined by, e.g., Affinity ELISA or assays as described herein) or its neutralization capability (as determined by e.g., Neutralization ELISA assays described herein, or similar assays) is at least 10 times as great, but optionally 50 times as great, 100, 250 or 500 times as great, or even at least 1000 times as great as the affinity or neutralization capability of the same for any other or other peptide or polypeptide.

The term “antigen binding domain” or “antigen binding region” refers to that portion of the specific binding agent (such as an antibody molecule) which contains the specific binding agent amino acid residues (or other moieties) that interact with an antigen and confer on the binding agent its specificity and affinity for the antigen. In an antibody, the antigen-binding domain is commonly referred to as the “complementarity-determining region, or CDR.”

The term “epitope” refers to that portion of any molecule capable of being recognized by and bound by a specific binding agent, e.g. an antibody, at one or more of the binding agent's antigen binding regions. Epitopes usually consist of chemically active surface groupings of molecules, such as for example, amino acids or carbohydrate side chains, and have specific three-dimensional structural characteristics as well as specific charge characteristics. Epitopes as used herein may be contiguous or non-contiguous. Moreover, epitopes may be mimetic in that they comprise a three dimensional structure that is identical to the epitope used to generate the antibody.

The term “antibody fragment” refers to a peptide or polypeptide which comprises less than a complete, intact antibody. Complete antibodies comprise two functionally independent parts or fragments: an antigen binding fragment known as “Fab,” and a carboxy terminal crystallizable fragment known as the “Fc” fragment. The Fab fragment includes the first constant domain from both the heavy and light chain together with the variable regions from both the heavy and light chains that bind the specific antigen. Each of the heavy and light chain variable regions includes three complementarity determining regions (CDRs) and framework amino acid residues which separate the individual CDRs. The Fc region comprises the second and third heavy chain constant regions and is involved in effector functions such as complement activation and attack by phagocytic cells. In some antibodies, the Fc and Fab regions are separated by an antibody “hinge region,” and depending on how the full length antibody is proteolytically cleaved, the hinge region may be associated with either the Fab or Fc fragment. For example, cleavage of an antibody with the protease papain results in the hinge region being associated with the resulting Fc fragment, while cleavage with the protease pepsin provides a fragment wherein the hinge is associated with both Fab fragments simultaneously. Because the two Fab fragments are in fact covalently linked following pepsin cleavage, the resulting fragment is termed the F(ab′)2 fragment.

An Fc domain may have a relatively long serum half-life, whereas a Fab is short-lived. See Capon et al., Nature, 337: 525-31 (1989). When expressed as part of a fusion protein, an Fc domain can impart longer half-life or incorporate such functions as Fc receptor binding, Protein A binding, complement fixation and perhaps even placental transfer into the protein to which it is fused. The Fc region may be a naturally occurring Fc region, or may be altered to improve certain qualities, such as therapeutic qualities or circulation time.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. The variable regions typically differ extensively in amino acid sequence even among antibodies of the same species. The variable region of an antibody typically determines the binding and specificity of each particular antibody for its particular antigen. The variability in sequence is concentrated in those regions referred to as complementarity-determining regions (CDRs), while the more highly conserved regions in the variable domain are called framework regions (FR). The CDRs of the light and heavy chains contain within them the amino acids which are largely responsible for the direct interaction of the antibody with antigen, however, amino acids in the FRs can affect antigen binding/recognition as discussed herein infra.

### Whole Body ImmunoPET Reveals Active Viral Reservoirs in SIV Infected ART-Treated Aviremic Macaques and Elite Controllers

The detection of viral reservoirs in the context of controlled HIV infection, either during ART or in elite controllers (EC), remains a challenge using current tools, and is limited to blood and biopsy tissues. A method is disclosed herein to capture total body simian immunodeficiency virus (SIV) replication using immunoPET (positron emission tomography) with computer tomography (CT). The administration of a PEG-modified, ⁶⁴Cu-labeled SW gp120-specific monoclonal antibody led to readily detectable signals in the gastrointestinal tract, lymphoid tissues and reproductive organs of viremic monkeys. Viral signals were markedly reduced in ART treated aviremic monkeys but clearly detectable in colon, select lymph nodes, and in the small bowel, nasal turbinates, the genital tract and lung. In elite controllers, virus was detected primarily in foci in the small bowel, select lymphoid areas and the male reproductive tract, all confirmed by qRT-PCR and immunohistochemistry. This real-time viral imaging in vivo is readily translatable to clinical studies of HIV.

Delineating viral replication in the context of generalized infections has traditionally relied on indirect measures, such as evaluating viral loads in plasma or via specific tissue biopsies. Such approaches have been valuable for the clinical management of viral infections such as HIV or Hepatitis C infections, although they generally do not identify the site or source of virus replication in vivo. In a small percentage of HIV-infected individuals, termed elite controllers (EC), virus replication is controlled to undetectable levels, and disease progression may be delayed for decades. Despite undetectable virus in the plasma, virus evolution continues to occur consistent with ongoing tissue-contained virus replication. It is important to identify tissue sites that can serve as viral reservoirs, so that the mechanisms by which such reservoirs are maintained can be identified, facilitating the development of strategies for eliminating them, particularly in individuals suppressed by highly active anti-retroviral treatment. Ideally, a method to identify such sites would be minimally invasive, specific, sensitive and amenable to repeat application. Here the application of whole body imaging to the detection and localization of sites of SIV infection in chronically infected, antiretroviral therapy (ART)-treated, as well as elite controller macaques is described.

A non-invasive, sensitive, immunoPET contrast agent and an approach to define the localization of SW infected tissue and free virus within individual live chronically-infected, ART-treated, and EC animals is disclosed. The method was repeated within the same animals (before and during ART) without any clinical adverse effect. In viremic animals infection was concentrated within the mucosa of the gut, reiterating that these tissues, are a major site of SIV replication.

However, discrete areas of virus replication, confirmed by qRT-PCR and IHC, were also observed in both nasal associated tissues (post-ART) and within the reproductive tract of male animals. Within chronically infected, aviremic, ART-treated as well as EC animals, the methodology was able to detect residual virus, specifically and in various tissues, corroborated by qRT-PCR data. Thus this approach provides the ability to identify novel areas of virus replication that may be difficult to sample, except at necropsy. It may also provide a powerful tool to monitor the kinetics of viral replication in tissues over time during the application of therapeutic approaches. With the current efforts towards HIV eradication, this method provides an important tool for the determination of organ specific efficacy of such approaches, crucial to the elimination of virally infected cells.

The detailed study of the cellular composition of these specific foci of infection combined with site specific drug metabolite levels aided by the ability to image these specific sites will likely be important to the development of directed therapies aimed at clearing infection from these sites in both controllers and individuals under HAART. Moreover, use of this technology during acute SW infection may provide improved delineation of the specific routes and kinetics of viral spread based on the mode of virus infection and allow the identification of stages at which interruption of infection may be targeted using prophylactic methods.

### Lentiviral Antigen Binding Agents and Uses

Certain lentiviral binding agents disclosed herein directly aid antimicrobial and vaccine development, assist in answering basic biological questions regarding the dynamics of lentivirus infections and transmission, and assist in the evaluation of the efficacy of a preventive vaccine which may restrict viral replication to the initial foyer of infection following mucosal exposure. Even though the development of highly active anti-retroviral therapy (HAART) has been a success, HIV-1 re-emerges following cessation of HAART. In addition, it is clear that low-level viremia persists even in persons on suppressive regimens for more than 7 years. Thus, these regimens do not eradicate HIV-1, and these results are indicative of long lived viral reservoirs, possibly within the various lymphoid and non-lymphoid tissues.

The precise delineation of such reservoirs is only possible post-necropsy, a high logistic hurdle in humans and a costly proposition in the macaque model, which additionally suffers from sampling issues. Through the contrast agents disclosed herein, reservoirs and their dynamics over time are identified in live animals and later on in humans during the course of the infection in real time. These agents assist in the development of antiviral therapies, imaging their spatial location within the body to eliminate infected cells, and indicate their effectiveness towards specific infected cell populations. Other benefits include the ability to evaluate the dose and time-dependence of an antiviral agent and its effect spatially given different infection routes, and evaluate the ability of the virus to develop resistance to an antiviral agent; all repeatedly for monitoring kinetics of viral replication and without sacrificing the animal.

In certain embodiments, this disclosure relates to immunoglobulin-based positron emission tomography (PET) contrast agent against viral envelope proteins, e.g., lentivirus such as HIV and SIV gp120. This binding agent specifically targets infected cells and virus in HIV and SIV infected humans and rhesus macaques and allows for whole body, non-invasive, quantitative interrogation of virally infected cells, tissue, and free virus as a function of time and space in living mammals.

In certain embodiments, imaging using PET may be combined with computed tomography (CT), scintigraphy, single-photon emission computed tomography, or combinations thereof for imaging to view virus replication, virus resurgence, virus response to antiretroviral treatment, as well as the initial dissemination during acute infection, which assists in therapeutic and vaccine monitoring. In certain embodiments, radioisotopes for imaging in single-photon emission computed tomography include ¹¹¹In, ¹²³I, ¹³¹I, and ^{99m}Tc. Radioisotopes with beta decay, alpha decay, and low energy electrons, may be used in therapeutic applications. In certain embodiments, the radioisotope is selected from ¹³¹I, ⁹⁰Y, ¹⁷⁷Lu, ¹⁸⁶Re, ¹⁸⁸Re, ⁶⁷Cu, ²¹¹At, ²¹²Bi, ²¹³Bi, ²²⁵Ac, ¹²⁵I, and ⁶⁷Ga.

### Imaging and Positron Emission Tomography (PET)

In certain embodiments, the disclosure relates to methods of imaging a pathogenic infection comprising, a) administering a tracer composition comprising a specific binding agent of disclosed herein to a subject; b) detecting pairs of gamma rays emitted by the positron-emitting radionuclide; and c) generating an image indicating a location of the positron-emitting radionuclide within an area of the subject.

PET uses a radioactive tracer that is labeled with a positron-emitting radionuclide and a PET camera for imaging the subject. Once the tracer is prepared and administered to the subject, the PET radionuclide decays in the body of the subject by positron emission. The collision of an emitted positron with a nearby electron produces two γ-rays that are separated by 180 degrees. Two scintillation detectors that are separated by 180 degrees transmit a coincident signal when they are achieved simultaneously. The photon energy that is absorbed by the detectors is typically converted to visible light and detected by photomultiplier tubes. The light signal is converted into an electrical current, which is proportional to the incident photon energy. The registered events are reconstructed into a three-dimensional image representing the spatial distribution of the radioactive source in the studied subject. See Zibo & Conti, Adv Drug Deliv Rev, 2010, 62(11):1031-51.

PET methods can be used to create image and take quantitative measurements. Co-registration of anatomical structures obtained from computed tomography (CT) allows one to evaluate regions of interest (ROIs). Changes in tissue radiotracer concentration can be measured over time. In certain embodiments, contemplated radiotracer are selected from ⁷¹As, ⁷²As, ⁷⁴As, ⁷⁶Br, ¹¹C, ^{34m}Cl, ⁵⁵Co, ⁶²Cu, ⁶⁴Cu, ¹⁸F, ⁵²Fe, ⁶⁶Ga, ⁶⁸Ga, ¹²⁴I, ⁵²Mn, ¹³N, ¹⁵O, ⁸²Rb, ^{94m}Tc, ⁸⁶Y, and ⁸⁹Zr. After radiolabeling, reaction mixtures are typically purified to separate the precursor and other reagents prior to administration to a subject. The tracer may be sterilized by sterile membrane filtration.

For ⁶⁴Cu, ⁶⁸Ga, and ⁸⁹Zr radiolabeling is typically conjugated through a chelating molecule. Contemplated chelating molecules include, but are not limited to, ethylenediaminetetraacetic acid (EDTA), diethylene triamine pentaacetic acid (DTPA), 1,4,7,10-tetraazadodecane-N,N′,N″,N′″-tetraacetic acid (DOTA), 1,4,8,11-tetraazacyclotetradecane-1,4,8,11-tetraacetic acid (TETA), 1,4,7-triazacyclononane-1,4,7-triacetic acid (NOTA), CB-DO2A, CB-TE2A, and AmBaSar.

Alkylation with ¹¹CH₃I or ¹¹CH₃OTf is typically used for introducing carbon-11 into a molecule. Palladium(0)-mediated Stille-type coupling reactions to create precursor aromatic stannanes are an option used in the synthesis of ¹¹C labeled molecules. Aliphatic nucleophilic substitution ¹⁸F reactions will displace a leaving group, such as a sulfonate (e.g., triflate, mesylate, tosylate or nosylate) or other halides (Cl, Br or I) to incorporated ¹⁸F into a molecule. [¹⁸F]KF.K₂₂₂ labeling procedure in aprotic solvents, ¹⁸F-TBAF in tertiary alcohols, or the chelation of ¹⁸F-aluminum fluoride (Al—¹⁸F) by NOTA may be used.

Antibody or other polypeptide labeling with ¹⁸F and ¹²⁴I may be done by the use of amine, thiol, or carboxylic acid reactive prosthetic groups. Prosthetic groups are molecules that can be activated and coupled to specific functional groups within the peptides, proteins, and antibodies (such as amino, carboxylate or sulfhydryl groups). Examples of prosthetic groups include, but not limited to 2-¹⁸F-Fluoroacetic acid; 2-¹⁸F-fluoropropionic acid; ¹⁸F-2,3,5,6-Tetrafluorophenyl pentafluorobenzoate; 4-Nitrophenyl 2-¹⁸F-fluoropropionate; ¹⁸F-SiFA-isothiocyanate; N-Succinimidyl 4-¹⁸F-fluorobenzoate; N-Succinimidyl 4-¹⁸F-fluoromethyl-benzoate; N-Succinimidyl 3-¹²⁴I-fluoromethyl-benzoate; N-succinimidyl 8-[(4′-¹⁸F-fluorobenzyl)amino]suberate; 1-[(4-¹⁸F-Fluoromethyl)benzoyl]-aminobutane-4-amine; Methyl 3-¹⁸F-Fluoro-5-nitrobenzimidate; 4-¹⁸F-Fluorophenacylbromide; 4-Azidophen-acyl-¹⁸F-fluoride; ¹⁸F-Pentafluorobenzaldehyde; N-(p-¹⁸F-Fluorophenyl) maleimide; m-Maleimido-N-(p-¹⁸F-fluorobenzyl)benzamide; N-[2-(4-¹⁸F-fluoro-benzamido)ethyl] maleimide; N-[6-(4-¹⁸F-fluorobenzyl-idene)aminooxyhexyl]maleimide; N-[4-[(4-¹⁸F-fluorobenzylidene)aminooxy]butyl]-maleimide; 1-[3-(2-¹⁸F-fluoropyridin-3-yloxy)propyl]pyrrole-2,5-dione; 4-¹⁸F-fluorobenzaldehyde-O-(2-2-[2-(pyrrol-2,5-dione-1-yl)ethoxy]-ethoxyethyl) oxime, ¹⁸F-FDG-maleimidehexyloxime; and 4-¹⁸F-Fluorobenzaldehyde.

### Antibodies and Antibody Mimetics

In certain embodiments, the disclosure contemplates specific binding agents that target pathogenic antigens, e.g., molecules on the exterior of viral particles and infected cells, envelope proteins, glycoproteins, saccharides, such as HIV gp120, that are antibodies or fragments or chimera, antibody mimetics, or aptamers.

Numerous methods known to those skilled in the art are available for obtaining antibodies or antigen-binding fragments thereof. For example, antibodies can be produced using recombinant DNA methods. See U.S. Pat. No. 4,816,567. Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

The modular structure of antibodies makes it possible to remove constant domains in order to reduce size and still retain antigen binding specificity. Engineered antibody fragments allow one to create antibody libraries. A single-chain antibody (scFv) is an antibody fragment where the variable domains of the heavy (VH) and light chains (VL) are combined with a flexible polypeptide linker. The scFv and Fab fragments are both monovalent binders but they can be engineered into multivalent binders to gain avidity effects. One exemplary method of making antibodies and fragments includes screening protein expression libraries, e.g., phage or ribosome display libraries. Phage display is described, for example, in U.S. Pat. No. 5,223,409.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. U.S. Pat. No. 7,064,244.

Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies. See U.S. Pat. No. 5,225,539. All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Computational methods may be utilized to generate fully human mAbs from nonhuman variable regions using information from the human germline repertoire. See U.S. Pat. Nos. 8,314,213, 7,930,107, 7,317,091, and Bernett et al., entitled, “Engineering fully human monoclonal antibodies from murine variable regions,” J Mol Biol. 2010, 396(5):1474-90.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the V_{H} and V_{L} sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

Thus, an embodiment of the present disclosure includes mutagenic strategies with the goal of increasing the affinity of an antibody for its target. These strategies include mutagenesis of the entire variable heavy and light chain, mutagenesis of the CDR regions only, mutagenesis of the consensus hypermutation sites within the CDRs, mutagenesis of framework regions, or any combination of these approaches (“mutagenesis” in this context could be random or site-directed). Definitive delineation of the CDR regions and identification of residues comprising the binding site of an antibody can be accomplished though solving the structure of the antibody in question, and the antibody-ligand complex, through techniques known to those skilled in the art, such as X-ray crystallography. Various methods based on analysis and characterization of such antibody crystal structures are known to those of skill in the art and can be employed, although not definitive, to approximate the CDR regions. Examples of such commonly used methods include the Kabat, Chothia, AbM and contact definitions.

The Kabat definition is based on the sequence variability and is the most commonly used definition to predict CDR regions. See Johnson and Wu, Nucleic Acids Res, 28: 214-8 (2000). The Chothia definition is based on the location of the structural loop regions. See Chothia et al., J Mol Biol, 196: 901-17 (1986); Chothia et al., Nature, 342: 877-83 (1989). The AbM definition is a compromise between the Kabat and Chothia definition. AbM is an integral suite of programs for antibody structure modeling produced by Oxford Molecular Group. See Martin et al., Proc Natl Acad Sci (USA) 86:9268-9272 (1989); Rees, et al., ABM™, a computer program for modeling variable regions of antibodies, Oxford, UK; Oxford Molecular, Ltd. The AbM suite models the tertiary structure of an antibody from primary sequencing using a combination of knowledge databases and ab initio methods. An additional definition, known as the contact definition, has been recently introduced. See MacCallum et al., J Mol Biol, 5:732-45 (1996). This definition is based on an analysis of the available complex crystal structures.

By convention, the CDR regions in the heavy chain are typically referred to as H1, H2 and H3 and are numbered sequentially in order counting from the amino terminus to the carboxy terminus. The CDR regions in the light chain are typically referred to as L1, L2 and L3 and are numbered sequentially in order counting from the amino terminus to the carboxy terminus. The CDR-H1 is approximately 10 to 12 residues in length and typically starts 4 residues after a Cys according to the Chothia and AbM definitions or typically 5 residues later according to the Kabat definition. The H1 is typically followed by a Trp, typically Trp-Val, but also Trp-Ile, or Trp-Ala. The length of H1 is approximately 10 to 12 residues according to the AbM definition while the Chothia definition excludes the last 4 residues.

The CDR-H2 typically starts 15 residues after the end of H1 according to the Kabat and AbM definition. The residues preceding H2 are typically Leu-Glu-Trp-Ile-Gly but there are a number of variations. H2 is typically followed by the amino acid sequence Lys/Arg-Leu/Ile/Val/Phe/Thr/Ala-Thr/Ser/Ile/Ala. According to the Kabat definition, the length of the H2 is approximately 16 to 19 residues where the AbM definition predicts the length to be typically 9 to 12 residues.

The CDR-H3 typically starts 33 residues after the end of H2 and is typically preceded by the amino acid sequence (typically Cys-Ala-Arg). The H3 is typically followed by the amino acid sequence-Gly. The length of H3 can be anywhere between 3 to 25 residues. The CDR-L1 typically starts at approximately residue 24 and will typically follow a Cys. The residue after the CDR-L1 is a Trp and will typically begin the sequence Trp-Tyr-Gln, Trp-Leu-Gln, Trp-Phe-Gln, or Trp-Tyr-Leu. The length of CDR-L1 is approximately 10 to 17 residues. The punitive CDR-L1 for the antibodies of the disclosure follows this pattern with a Cys residue followed by 15 amino acids then Trp-Tyr-Gln.

The CDR-L2 starts approximately 16 residues after the end of L1. It will generally follow residues Ile-Tyr, Val-Tyr, Ile-Lys or Ile-Phe. The length of CDR-L2 is approximately 7 residues. The CDR-L3 typically starts 33 residues after the end of L2 and typically follows a Cys. L3 is typically followed by the amino acid sequence Phe-Gly-XXX-Gly. The length of L3 is approximately 7 to 11 residues.

Various methods for modifying antibodies have been described in the art. For example, U.S. Pat. No. 5,530,101 (to Queen et al.) describes methods to produce humanized antibodies wherein the sequence of the humanized immunoglobulin heavy chain variable region framework is 65% to 95% identical to the sequence of the donor immunoglobulin heavy chain variable region framework. Each humanized immunoglobulin chain will usually comprise, in addition to the CDRs, amino acids from the donor immunoglobulin framework that are, e.g., capable of interacting with the CDRs to affect binding affinity, such as one or more amino acids which are immediately adjacent to a CDR in the donor immunoglobulin or those within about 3 angstroms as predicted by molecular modeling. The heavy and light chains may each be designed by using any one or all of various position criteria. When combined into an intact antibody, the humanized immunoglobulins of the present disclosure will be substantially non-immunogenic in humans and retain substantially the same affinity as the donor immunoglobulin to the antigen, such as a protein or other compound containing an epitope. See also, related methods in U.S. Pat. No. 5,693,761 to Queen, et al. (“Polynucleotides encoding improved humanized immunoglobulins”); U.S. Pat. No. 5,693,762 to Queen, et al. (“Humanized Immunoglobulins”); U.S. Pat. No. 5,585,089 to Queen, et al. (“Humanized Immunoglobulins”).

In one example, U.S. Pat. No. 5,565,332 to Hoogenboom et al. (“Production of chimeric antibodies-a combinatorial approach”) describes methods for the production of antibodies, and antibody fragments which have similar binding specificity as a parent antibody but which have increased human characteristics. Humanized antibodies are obtained by chain shuffling, using, for example, phage display technology, and a polypeptide comprising a heavy or light chain variable domain of a non-human antibody specific for an antigen of interest is combined with a repertoire of human complementary (light or heavy) chain variable domains. Hybrid pairings that are specific for the antigen of interest are identified and human chains from the selected pairings are combined with a repertoire of human complementary variable domains (heavy or light). In another embodiment, a component of a CDR from a non-human antibody is combined with a repertoire of component parts of CDRs from human antibodies. From the resulting library of antibody polypeptide dimers, hybrids are selected and used in a second humanizing shuffling step. Alternatively, this second step is eliminated if the hybrid is already of sufficient human character to be of therapeutic value. Methods of modification to increase human character are also described. See also Winter, FEBS Letts 430:92-92 (1998).

As another example, U.S. Pat. No. 6,054,297 to Carter et al. describes a method for making humanized antibodies by substituting a CDR amino acid sequence for the corresponding human CDR amino acid sequence and/or substituting a FR amino acid sequence for the corresponding human FR amino acid sequences.

As another example, U.S. Pat. No. 5,766,886 to Studnicka et al. (“Modified antibody variable domains”) describes methods for identifying the amino acid residues of an antibody variable domain which may be modified without diminishing the native affinity of the antigen binding domain while reducing its immunogenicity with respect to a heterologous species and methods for preparing these modified antibody variable domains which are useful for administration to heterologous species. See also U.S. Pat. No. 5,869,619 to Studnicka. Modification of an antibody by any of the methods known in the art is typically designed to achieve increased binding affinity for an antigen and/or reduce immunogenicity of the antibody in the recipient. In one approach, humanized antibodies can be modified to eliminate glycosylation sites in order to increase affinity of the antibody for its cognate antigen. See Co et al., Mol Immunol 30:1361-1367 (1993). Techniques such as “reshaping,” “hyperchimerization,” and “veneering/resurfacing” have produced humanized antibodies with greater therapeutic potential. See Vaswami et al., Annals of Allergy, Asthma, & Immunol 81:105 (1998); Roguska et al., Prot Engineer 9:895-904 (1996). See also U.S. Pat. No. 6,072,035 to Hardman et al., 2000, which describes methods for reshaping antibodies. While these techniques diminish antibody immunogenicity by reducing the number of foreign residues, they do not prevent anti-idiotypic and anti-allotypic responses following repeated administration of the antibodies. Alternatives to these methods for reducing immunogenicity are described in Gilliland et al., J Immunol 62(6): 3663-71 (1999).

In certain instances, humanizing antibodies result in a loss of antigen binding capacity. It is therefore preferable to “back mutate” the humanized antibody to include one or more of the amino acid residues found in the original (most often rodent) antibody in an attempt to restore binding affinity of the antibody. See, for example, Saldanha et al., Mol Immunol 36:709-19 (1999).

Antibody mimetics or engineered affinity proteins are polypeptide based targeting moieties that can specifically bind to targets but are not specifically derived from antibody V_{H} and V_{L} sequences. Typically, a protein motif is recognized to be conserved among a number of proteins. One can artificially create libraries of these polypeptides with amino acid diversity and screen them for binding to targets through phage, yeast, bacterial display systems, cell-free selections, and non-display systems. See Gronwall & Stahl, J Biotechnology, 2009, 140(3-4), 254-269, hereby incorporated by reference in its entirety. Antibody mimetics include affibody molecules, affilins, affitins, anticalins, avimers, darpins, fynomers, kunitz domain peptides, and monobodies.

Affibody molecules are based on a protein domain derived from staphylococcal protein A (SPA). SPA protein domain denoted Z consists of three a-helices forming a bundle structure and binds the Fc portion of human IgG1. A combinatorial library may be created by varying surface exposed residues involved in the native interaction with Fc. Affinity proteins can be isolated from the library by phage display selection technology.

Monobodies, sometimes referred to as adnectins, are antibody mimics based on the scaffold of the fibronectin type III domain (FN3). See Koide et al., Methods Mol. Biol. 2007, 352: 95-109, hereby incorporated by reference in its entirety. FN3 is a 10 kDa, β-sheet domain, that resembles the V_{H} domain of an antibody with three distinct CDR-like loops, but lack disulfide bonds. FN3 libraries with randomized loops have successfully generated binders via phage display (M13 gene 3, gene 8; T7), mRNA display, yeast display and yeast two-hybrid systems. See Bloom & Calabro, Drug Discovery Today, 2009, 14(19-20):949-955, hereby incorporated by reference in its entirety.

Anticalins, sometimes referred to as lipocalins, are a group of proteins characterized by a structurally conserved rigid β-barrel structure and four flexible loops. The variable loop structures form an entry to a ligand-binding cavity. Several libraries have been constructed based on natural human lipocalins, i.e., ApoD, NGAL, and Tlc. Anticalins have been generated for targeting the cytotoxic T-lymphocyte antigen-4 (CTLA-4). See Skerra, FEBS J., 275 (2008), pp. 2677-2683, and Binder et al., J Mol Biol., 2010, 400(4):783-802., both hereby incorporated by reference in their entirety.

The ankyrin repeat (AR) protein is composed repeat domains consisting of a β-turn followed by two α-helices. Natural ankyrin repeat proteins normally consist of four to six repeats. The ankyrin repeats form a basis for darpins (designed ankyrin repeat protein) which is a scaffold comprised of repeats of an artificial consensus ankyrin repeat domain. Combinatorial libraries have been created by randomizing residues in one repeat domain. Different numbers of the generated repeat modules can be connected together and flanked on each side by a capping repeat. The darpin libraries are typically denoted NxC, where N stands for the N-terminal capping unit, C stands for the C-terminal capping domain and x for the number of library repeat domains, typically between two to four. See Zahnd et al., J. Mol. Biol., 2007, 369:1015-1028, hereby incorporated by reference in its entirety.

Aptamers refer to specific binding agents identified from random proteins or nucleic acids libraries. Peptide aptamers have been selected from random loop libraries displayed on TrxA. See Borghouts et al., Expert Opin. Biol. Ther., 2005, 5:783-797, hereby incorporated by reference in its entirety. SELEX (“Systematic Evolution of Ligands by Exponential Enrichment”) is a combinatorial chemistry technique for producing oligonucleotides of either single-stranded DNA or RNA that specifically bind to a target. Standard details on generating nucleic acid aptamers can be found in U.S. Pat. No. 5,475,096, and U.S. Pat. No. 5,270,163. The SELEX process provides a class of products which are referred to as nucleic acid ligands or aptamers, which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

### Viruses and Viral Specific Binding Agents

In some embodiments, the disclosure relates to methods of treating a viral infection comprising administering a viral specific binding agent disclosed herein conjugated to a molecule with a positron-emitting radionuclide, ⁶⁴Cu, to a subject that is diagnosed with, suspected of, or exhibiting symptoms of a viral infection. Typically the specific binding agent has affinity for a viral protein, glycoprotein, saccharide, or polysaccharide displayed on the exterior of a viral particle.

Viruses are infectious agents that can typically replicate inside the living cells of organisms. Virus particles (virions) usually consist of nucleic acids, a protein coat, and in some cases lipids that surrounds the protein coat. The shapes of viruses range from simple helical and icosahedral forms to more complex structures. Virally coded protein subunits will self-assemble to form a capsid, generally requiring the presence of the virus genome. Complex viruses code for proteins that assist in the construction of their capsid. Proteins associated with nucleic acid are known as nucleoproteins, and the association of viral capsid proteins with viral nucleic acid is called a nucleocapsid.

A virus has either DNA or RNA genes and is called a DNA virus or a RNA virus respectively. A viral genome is either single-stranded or double-stranded. Some viruses contain a genome that is partially double-stranded and partially single-stranded. For viruses with RNA or single-stranded DNA, the strands are said to be either positive-sense (called the plus-strand) or negative-sense (called the minus-strand), depending on whether it is complementary to the viral messenger RNA (mRNA). Positive-sense viral RNA is identical to viral mRNA and thus can be immediately translated by the host cell. Negative-sense viral RNA is complementary to mRNA and thus is to be converted to positive-sense RNA by an RNA polymerase before translation. DNA nomenclature is similar to RNA nomenclature, in that the coding strand for the viral mRNA is complementary to it (negative), and the non-coding strand is a copy of it (positive). Antigenic shift, or re-assortment, can result in novel strains. Viruses undergo genetic change by several mechanisms. These include a process called genetic drift where individual bases in the DNA or RNA mutate to other bases. Antigenic shift occurs when there is a major change in the genome of the virus. This can be a result of recombination or re-assortment. RNA viruses often exist as quasi-species or swarms of viruses of the same species but with slightly different genome nucleoside sequences.

The Baltimore classification of viruses is based on the mechanism of mRNA production. Viruses must generate mRNAs from their genomes to produce proteins and replicate themselves, but different mechanisms are used to achieve this. Viral genomes may be single-stranded (ss) or double-stranded (ds), RNA or DNA, and may or may not use reverse transcriptase (RT). Additionally, ssRNA viruses may be either sense (plus) or antisense (minus). This classification places viruses into seven groups: I, dsDNA viruses (e.g. adenoviruses, herpesviruses, poxviruses); II, ssDNA viruses (plus)sense DNA (e.g. parvoviruses); III, dsRNA viruses (e.g. reoviruses); IV, (plus)ssRNA viruses (plus)sense RNA (e.g. picornaviruses, togaviruses); V, (minus)ssRNA viruses (minus)sense RNA (e.g. orthomyxoviruses, Rhabdoviruses); VI, ssRNA-RT viruses (plus)sense RNA with DNA intermediate in life-cycle (e.g. retroviruses); and VII, dsDNA-RT viruses (e.g. hepadnaviruses).

In certain embodiments, the subject is diagnosed to have a virus by nucleic acid detection or viral antigen detection.

In certain embodiments, the disclosure relates to methods of imaging, treating or preventing an HIV infection comprising administering a specific binding agent with affinity for HIV gp120 conjugated to a radioisotope. Typically, the binding agent is an antibody that binds gp120 and the radioisotope, e.g., ⁶⁴ Cu decays in manner that disrupts, breaks-down, or facilitates the breakdown or replication of the HIV particle and viral nucleic acids. The radiolabeled antibody for gp120 may be administered in combination with other anti-viral agents.

HIV is a lentivirus (a member of the retrovirus family) that causes acquired immunodeficiency syndrome (AIDS). Lentiviruses are transmitted as single-stranded, positive-sense, enveloped RNA viruses. Upon entry of the target cell, the viral RNA genome is converted to double-stranded DNA by a virally encoded reverse transcriptase. This viral DNA is then integrated into the cellular DNA by a virally encoded integrase, along with host cellular co-factors. There are two species of HIV. HIV-1 is sometimes termed LAV or HTLV-III. HIV infects primarily vital cells in the human immune system such as helper T cells (CD4+ T cells), macrophages, and dendritic cells. HIV infection leads to low levels of CD4+ T cells. When CD4+ T cell numbers decline below a critical level, cell-mediated immunity is lost, and the body becomes progressively more susceptible to other viral or bacterial infections. Subjects with HIV typically develop malignancies associated with the progressive failure of the immune system.

The viral envelope is composed of two layers of phospholipids taken from the membrane of a human cell when a newly formed virus particle buds from the cell. Embedded in the viral envelope are proteins from the host cell and a HIV protein known as Env. Env contains glycoproteins gp120, and gp41. The RNA genome consists of structural landmarks (LTR, TAR, RRE, PE, SLIP, CRS, and INS) and nine genes (gag, pol, and env, tat, rev, nef, vif, vpr, vpu, and sometimes a tenth tev, which is a fusion of tat env and rev) encoding 19 proteins. Three of these genes, gag, pol, and env, contain information needed to make the structural proteins for new virus particles.

GP120 (or gp120) is a glycoprotein exposed on the surface of the HIV virion as a spike, composed of three copies of the gp120 exterior envelope glycoprotein and three gp41 transmembrane glycoprotein molecules. The gp120 protein is divided into five conserved (C1-C5) and five variable (V1-V5) segments. Gp120 is important for virus entry into host cells, e.g., helper T-cells, by facilitation attachment to specific cell surface receptors such as CD4 receptor, CCR5, and CXCR4. Although gp120 is the primary target of antibodies elicited during natural infection, human vaccine candidates for HIV-1 have not been able to elicit broadly immune eradicating antibodies. HIV-1 gp120 is believed to evade clearance from the host immune system due to the presence of variable loops, N-linked glycosylation, conformational flexibility, and sanctuaries for hibernation.

In certain embodiments, the disclosure relates to methods treating or preventing an HIV infection comprising administering a viral specific binding agent conjugated to a radioisotope with affinity for HIV gp120. Typically, the binding agent is an antibody that binds gp120 and the radioisotope, e.g., ⁶⁴Cu, decays in manner that eradicates HIV or cells containing HIV particles. The radiolabeled antibody for gp120 may be administered in combination with other anti-viral agents.

With regard to any of the embodiments disclosed herein, the binding agent may be a human antibody, humanized antibody, or chimera. The agent may be CD4-binding site (CD4BS) antibodies, partially neutralizing, or non-neutralizing antibody.

In certain embodiments, the binding agent may be human or humanized IgG or F_{ab} fragment of CH103 as provide in Liao et al., Nature, doi:10.1038/nature12053.

In certain embodiments, the binding agent may be human or humanized IgG or F_{ab} fragment of PG V04 as provided in Falkowska et al., J. Virol, 2012, 86:4394-4403.

In certain embodiments, the binding agent may be human or humanized IgG or F_{ab} fragment of PGT-127, PGT-128, PGT-130, and PGT-131 as provided in Pejchal R, et al. Science, 2011, 334:1097-1103.

In certain embodiments, the binding agent may be human or humanized IgG or F_{ab} fragment of CH01, CH02, CH03, and CH04 as provided in Bonsignori et al., J. Virol, 2011, 85:9998-10009.

In certain embodiments, the binding agent may be human or humanized IgG or F_{ab} fragment of human antibody 2909 as provided in Changela et al., J. Virol, 2011, 85:2524-2535.

In certain embodiments, the binding agent may be human or humanized IgG or F_{ab} fragment of VRC01, VRC02, and VRC03 as provided in Wu et al., Science, 2010 329:856-861.

In certain embodiments, the binding agent may be human or humanized IgG or F_{ab} fragment of HJ16, HGN194 and HK20 as provided in Corti et al., PLoS One, 2010, 5:e8805.

In certain embodiments, the binding agent may be human or humanized IgG or F_{ab} fragment of PG9 or PG16 as provided in Walker et al., Science, 2009, 326:285-289.

In certain embodiments, the binding agent may be human or humanized IgG or F_{ab} fragment of 22A, 171C2, 71B7, 36D5, 31C7, 8H1, 189D5, 77D6, 3E9, 4B11, 5B11, 7D3, 8C7, 11F2, and 17A11 as provided in Edinger, A. L., et al., J Virol, 2000, 74(17): 7922-35.

In certain embodiments, the binding agent may be human or humanized IgG or F_{ab} fragment of 2G12 as provided in Trkola et al., J Virol, 1996, 70:1100-1108.

In certain embodiments, the binding agent may be IgG or F_{ab} fragment of b12, b13, ml 8, and F105 as provided in Burton et al., Science 266, 1024 (1994).

In certain embodiments, the binding agent may be human or humanized IgG or F_{ab} fragment of 447-52D as provided in Gorny et al, J Virol, 1992, 66:7538-42.

In certain embodiments, the binding agent may be soluble versions of the CD4 receptor, including a monomeric four-domain version (sCD4, 1 mer) as provided in Deen et al., Nature, 331, 82 (1988).

In certain embodiments, the binding agent may be soluble versions of the CD4 receptor such as a dimeric immunoglobulin chimera (CD4-IgG, 2 mer), as provided in Traunecker et al., Nature, 339, 68 (1989).

In certain embodiments, the binding agent may be soluble versions of the CD4 receptor such as a dodecameric version (CD4 dodecamer, 12 mer) as provided in Arthos et al., J. Biol. Chem. 277, 11456.

HIV-1 diagnosis is typically done with antibodies in an ELISA, Western blot, or immunoaffinity assays or by nucleic acid testing (e.g., viral RNA or DNA amplification).

HIV is sometimes treated with HAART a combination of antiviral agent, e.g., two nucleoside-analogue reverse transcription inhibitors and one non-nucleoside-analogue reverse transcription inhibitor or protease inhibitor. The three drug combination is commonly known as a triple cocktail.

In certain embodiments, the disclosure relates to treating a subject diagnosed with HIV by administering a viral specific binding agent conjugated to a radioisotope in combination with two nucleoside-analogue reverse transcription inhibitors and one non-nucleoside-analogue reverse transcription inhibitor or protease inhibitor.

In certain embodiments, the disclosure relates to treating a subject by administering a viral specific binding agent conjugated to a radioisotope, emtricitabine, tenofovir, and efavirenz.

In certain embodiments, the disclosure relates to treating a subject by administering a viral specific binding agent conjugated to a radioisotope, emtricitabine, tenofovir and raltegravir.

In certain embodiments, the disclosure relates to treating a subject by administering a viral specific binding agent conjugated to a radioisotope, emtricitabine, tenofovir, ritonavir and darunavir.

In certain embodiments, the disclosure relates to treating a subject by administering a viral specific binding agent conjugated to a radioisotope, emtricitabine, tenofovir, ritonavir and atazanavir.

Banana lectin (BanLec or BanLec-1) is one of the predominant proteins in the pulp of ripe bananas and has binding specificity for mannose and mannose-containing oligosaccharides. BanLec binds to the HIV-1 envelope protein gp120. In certain embodiments, the disclosure relates to treating viral infections, such as HIV, by administering a banana lectin conjugated to a radioisotope optionally in combination with other antiviral agents.

### Combination Therapies

In some embodiments, the disclosure relates to treating a viral infection by administering a specific binding agent with affinity for a viral antigen conjugated to a radioisotope in combination with a second antiviral agent. In further embodiments, specific binding agent with affinity for a viral antigen conjugated to a radioisotope is administered in combination with one or more of the following agents: abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, elvitegravir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate (TAF), tipranavir, trifluridine, trizivir, tromantadine, truvada, valaciclovir, valganciclovir, vicriviroc, vidarabine, viramidine, zalcitabine, zanamivir, or zidovudine, 2-(3-chloro-4-fluorobenzyl)-8-ethyl-10-hydroxy-N,6-dimethyl-1,9-dioxo-1,2,6,7,8,9 hexahydropyrazino[1′,2′:1,5]pyrrolo[2,3-d]pyridazine-4-carboxamide (MK-2048), salts, and combinations thereof.

Antiviral agents include, but are not limited to, protease inhibitors (PIs), integrate inhibitors, entry inhibitors (fusion inhibitors), maturation inhibitors, and reverse transcriptase inhibitors (anti-retrovirals). Combinations of antiviral agents create multiple obstacles to viral replication, i.e., to keep the number of offspring low and reduce the possibility of a superior mutation. If a mutation that conveys resistance to one of the agents being taken arises, the other agents continue to suppress reproduction of that mutation. For example, a single anti-retroviral agent has not been demonstrated to suppress an HIV infection for long. These agents are typically taken in combinations in order to have a lasting effect. As a result, the standard of care is to use combinations of anti-retrovirals.

Reverse transcribing viruses replicate using reverse transcription, i.e., the formation of DNA from an RNA template. Retroviruses often integrate the DNA produced by reverse transcription into the host genome. They are susceptible to antiviral drugs that inhibit the reverse transcriptase enzyme. In certain embodiments the disclosure relates to methods of treating viral infections by administering a specific binding agent with affinity for a viral antigen conjugated to a radioisotope, and a retroviral agent such as nucleoside and nucleotide reverse transcriptase inhibitors (NRTI) and/or a non-nucleoside reverse transcriptase inhibitors (NNRTI). Examples of nucleoside reverse transcriptase inhibitors include zidovudine, didanosine, zalcitabine, stavudine, lamivudine, abacavir, emtricitabine, entecavir, apricitabine. Examples of nucleotide reverse transcriptase inhibitors include tenofovir and adefovir. Examples of non-nucleoside reverse transcriptase inhibitors include efavirenz, nevirapine, delavirdine, and etravirine.

In certain embodiments, the disclosure relates to methods of treating a viral infection by administering a specific binding agent with affinity for a viral antigen conjugated to a radioisotope in combination with an antiviral drug, e.g., 2′,3′-dideoxyinosine and a cytostatic agent, e.g., hydroxyurea.

Human immunoglobulin G (IgG) antibodies are believed to have opsonizing and neutralizing effects against certain viruses. IgG is sometimes administered to a subject diagnosed with immune thrombocytopenic purpura (ITP) secondary to a viral infection since certain viruses such as, HIV and hepatitis, cause ITP. In certain embodiments, the disclosure relates to methods of treating or preventing viral infections comprising administering a specific binding agent with affinity for a viral antigen in combination with an immunoglobulin to a subject. IgG is typically manufactured from large pools of human plasma that are screened to reduce the risk of undesired virus transmission. The Fc and Fab functions of the IgG molecule are usually retained. Therapeutic IgGs include Privigen, Hizentra, and WinRho. WinRho is an immunoglobulin (IgG) fraction containing antibodies to the Rho(D) antigen (D antigen). The antibodies have been shown to increase platelet counts in Rho(D) positive subjects with ITP. The mechanism is thought to be due to the formation of anti-Rho(D) (anti-D)-coated RBC complexes resulting in Fc receptor blockade, thus sparing antibody-coated platelets.

### Methods of Making Specific Binding Agents

Specific binding agents of the present disclosure that are proteins can be prepared by chemical synthesis in solution or on a solid support in accordance with conventional techniques. The current limit for solid phase synthesis is about 85-100 amino acids in length. However, chemical synthesis techniques can often be used to chemically ligate a series of smaller peptides to generate full length polypeptides. Various automatic synthesizers are commercially available and can be used in accordance with known protocols. See, for example, Stewart and Young, Solid Phase Peptide Synthesis, 2d. ed., Pierce Chemical Co., (1984); Tam et al., J Am Chem Soc, 105:6442, (1983); Merrifield, Science, 232:341-347, (1986); and Barany and Merrifield, The Peptides, Gross and Meienhofer, eds, Academic Press, New York, 1-284; Barany et al., Int. J. Peptide Protein Res., 30, 705-739 (1987); and U.S. Pat. No. 5,424,398), each incorporated herein by reference.

Solid phase peptide synthesis methods use a copoly(styrene-divinylbenzene) containing 0.1-1.0 mM amines/g polymer. These methods for peptide synthesis use butyloxycarbonyl (t-BOC) or 9-fluorenylmethyloxy-carbonyl(FMOC) protection of alpha-amino groups. Both methods involve stepwise syntheses whereby a single amino acid is added at each step starting from the C-terminus of the peptide (See, Coligan et al., Current Protocols in Immunology, Wiley Interscience, 1991, Unit 9). On completion of chemical synthesis, the synthetic peptide can be deprotected to remove the t-BOC or FMOC amino acid blocking groups and cleaved from the polymer by treatment with acid at reduced temperature (e.g., liquid HF-10% anisole for about 0.25 to about 1 hour at 0 degree C.). After evaporation of the reagents, the specific binding agent peptides are extracted from the polymer with 1% acetic acid solution that is then lyophilized to yield the crude material. This can normally be purified by such techniques as gel filtration on Sephadex G-15 using 5% acetic acid as a solvent. Lyophilization of appropriate fractions of the column will yield the homogeneous specific binding agent peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation. Chemical synthesis of anti-gp120 antibodies, derivatives, variants, and fragments thereof, as well as other protein-based gp120 binding agents permits incorporation of non-naturally occurring amino acids into the agent.

Recombinant DNA techniques are a convenient method for preparing full length antibodies and other large proteinaceous specific binding agents of the present disclosure, or fragments thereof. A cDNA molecule encoding the antibody or fragment may be inserted into an expression vector, which can in turn be inserted into a host cell for production of the antibody or fragment. It is understood that the cDNAs encoding such antibodies may be modified to vary from the “original” cDNA (translated from the mRNA) to provide for codon degeneracy or to permit codon preference usage in various host cells.

Where it is desirable to obtain Fab molecules or CDRs that are related to the original antibody molecule, one can screen a suitable library (phage display library; lymphocyte library, etc.) using standard techniques to identify and clone related Fabs/CDRs. Probes used for such screening may be full length or truncated Fab probes encoding the Fab portion of the original antibody, probes against one or more CDRs from the Fab portion of the original antibody, or other suitable probes. Where DNA fragments are used as probes, typical hybridization conditions are those such as set forth in Ausubel et al. (Current Protocols in Molecular Biology, Current Protocols Press [1994]). After hybridization, the probed blot can be washed at a suitable stringency, depending on such factors as probe size, expected homology of probe to clone, the type of library being screened, and the number of clones being screened. Examples of high stringency screening are 0.1 times SSC, and 0.1 percent SDS at a temperature between 50-65 degree C.

A variety of expression vector/host systems may be utilized to contain and express the polynucleotide molecules encoding the specific binding agent polypeptides of the disclosure. These systems include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems; mammalian cells transformed with pseudotyped lentiviral expression vectors.

Mammalian cells that are useful in recombinant specific binding agent protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells, as well as hybridoma cell lines as described herein. Mammalian cells are preferred for preparation of those specific binding agents such as antibodies and antibody fragments that are typically glycosylated and require proper refolding for activity. Preferred mammalian cells include CHO cells, 293, hybridoma cells, and myeloid cells.

Some exemplary protocols for the recombinant expression of the specific binding agent proteins are described herein below.

The term “expression vector” refers to a plasmid, phage, virus or vector, for expressing a polypeptide from a DNA (RNA) sequence. An expression vector can comprise a transcriptional unit comprising an assembly of (1) a genetic element or elements having a regulatory role in gene expression, for example, promoters or enhancers, (2) a structural or sequence that encodes the binding agent which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant specific binding agent protein is expressed without a leader or transport sequence, it may include an amino terminal methionine residue. This residue may or may not be subsequently cleaved from the expressed recombinant protein to provide a final specific binding agent product.

For example, the specific binding agents may be recombinantly expressed in yeast using a commercially available expression system, e.g., the Pichia Expression System (Invitrogen, San Diego, Calif), following the manufacturer's instructions. This system also relies on the pre-pro-alpha sequence to direct secretion, but transcription of the insert is driven by the alcohol oxidase (AOX1) promoter upon induction by methanol. The secreted specific binding agent peptide is purified from the yeast growth medium by, e.g., the methods used to purify the peptide from bacterial and mammalian cell supernatants.

Alternatively, the cDNA encoding the specific binding agent peptide may be cloned into the baculovirus expression vector pVL1393 (PharMingen, San Diego, Calif). This vector can be used according to the manufacturer's directions (PharMingen) to infect Spodoptera frugiperda cells in sF9 protein-free media and to produce recombinant protein. The specific binding agent protein can be purified and concentrated from the media using a heparin-Sepharose column (Pharmacia).

Alternatively, the peptide may be expressed in an insect system. Insect systems for protein expression are well known to those of skill in the art. In one such system, Autographa californica nuclear polyhedrosis virus (AcNPV) can be used as a vector to express foreign genes in Spodoptera frugiperda cells or in Trichoplusia larvae. The specific binding agent peptide coding sequence can be cloned into a nonessential region of the virus, such as the polyhedrin gene, and placed under control of the polyhedrin promoter. Successful insertion of the specific binding agent peptide will render the polyhedrin gene inactive and produce recombinant virus lacking coat protein coat. The recombinant viruses can be used to infect S. frugiperda cells or Trichoplusia larvae in which peptide is expressed [Smith et al., J Virol 46: 584 (1983); Engelhard et al., Proc Nat Acad Sci (USA) 91: 3224-7 (1994)].

In another example, the DNA sequence encoding the specific binding agent peptide can be amplified by PCR and cloned into an appropriate vector for example, pGEX-3X (Pharmacia). The pGEX vector is designed to produce a fusion protein comprising glutathione-S-transferase (GST), encoded by the vector, and a specific binding agent protein encoded by a DNA fragment inserted into the vector's cloning site. The primers for the PCR can be generated to include for example, an appropriate cleavage site. Where the specific binding agent fusion moiety is used solely to facilitate expression or is otherwise not desirable as an attachment to the peptide of interest, the recombinant specific binding agent fusion protein may then be cleaved from the GST portion of the fusion protein. The pGEX-3X/specific binding agent peptide construct is transformed into E. coli XL-1 Blue cells (Stratagene, La Jolla Calif), and individual transformants isolated and grown. Plasmid DNA from individual transformants can be purified and partially sequenced using an automated sequencer to confirm the presence of the desired specific binding agent encoding nucleic acid insert in the proper orientation.

Expression of polynucleotides encoding antibodies and fragments thereof using the recombinant systems described above may result in production of antibodies or fragments thereof that must be “re-folded” (to properly create various disulphide bridges) in order to be biologically active. Typical refolding procedures for such antibodies are set forth in the Examples herein and in the following section.

Specific binding agents made in bacterial cells may be produced as an insoluble inclusion body in the bacteria, can be purified as follows. Host cells can be sacrificed by centrifugation; washed in 0.15 M NaCl, 10 mM Tris, pH 8, 1 mM EDTA; and treated with 0.1 mg/ml lysozyme (Sigma, St. Louis, Mo.) for 15 minutes at room temperature. The lysate can be cleared by sonication, and cell debris can be pelleted by centrifugation for 10 minutes at 12,000.times·g. The specific binding agent-containing pellet can be resuspended in 50 mM Tris, pH 8, and 10 mM EDTA, layered over 50% glycerol, and centrifuged for 30 min. at 6000 times g. The pellet can be re-suspended in standard phosphate buffered saline solution (PBS) free of Mg and Ca. The specific binding agent can be further purified by fractionating the resuspended pellet in a denaturing SDS polyacrylamide gel (Sambrook et al., supra). The gel can be soaked in 0.4 M KCl to visualize the protein, which can be excised and electroeluted in gel-running buffer lacking SDS. If the GST fusion protein is produced in bacteria, as a soluble protein, it can be purified using the GST Purification Module (Pharmacia).

Host cell strains can be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Different host cells such as CHO, HeLa, MDCK, 293, WI38, as well as hybridoma cell lines, and the like have specific cellular machinery and characteristic mechanisms for such post-translational activities and can be chosen to ensure the correct modification and processing of the introduced, foreign protein.

A number of selection systems can be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for DHFR which confers resistance to methotrexate; gpt which confers resistance to mycophenolic acid; neo which confers resistance to the aminoglycoside G418 and confers resistance to chlorsulfuron; and hygro which that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, beta-glucuronidase and its substrate, GUS, and luciferase and its substrate, luciferin.

In some cases, the specific binding agents produced using procedures described above may need to be “refolded” and oxidized into a proper tertiary structure and generating di-sulfide linkages in order to be biologically active. Refolding can be accomplished using a number of procedures well known in the art. Such methods include, for example, exposing the solubilized polypeptide agent to a pH usually above 7 in the presence of a chaotropic agent. The selection of chaotrope is similar to the choices used for inclusion body solubilization, however a chaotrope is typically used at a lower concentration. An exemplary chaotropic agent is guanidine. In most cases, the refolding/oxidation solution will also contain a reducing agent plus its oxidized form in a specific ratio to generate a particular redox potential which allows for dusykfide shuffling to occur for the formation of cysteine bridges. Some commonly used redox couples include cysteine/cystamine, glutathione/dithiobisGSH, cupric chloride, dithiothreitol DTT/dithiane DTT, and 2-mercaptoethanol (bME)/dithio-bME. In many instances, a co-solvent may be used to increase the efficiency of the refolding Commonly used co-solvents include glycerol, polyethylene glycol of various molecular weights, and arginine.

It will be desirable to purify specific binding agent proteins or variants thereof of the present disclosure. Protein purification techniques are well known to those of skill in the art. These techniques involve, at one level, the crude fractionation of the polypeptide and non-polypeptide fractions. Having separated the specific binding agent polypeptide from other proteins, the polypeptide of interest can be further purified using chromatographic and electrophoretic techniques to achieve partial or complete purification (or purification to homogeneity). Analytical methods particularly suited to the preparation of a pure specific binding agent peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present disclosure concerns the purification, and in particular embodiments, the substantial purification, of an encoded specific binding agent protein or peptide. The term “purified specific binding agent protein or peptide” as used herein, is intended to refer to a composition, isolatable from other components, wherein the specific binding agent protein or peptide is purified to any degree relative to its naturally-obtainable state. A purified specific binding agent protein or peptide therefore also refers to a specific binding agent protein or peptide, free from the environment in which it may naturally occur.

Generally, “purified” will refer to a specific binding agent composition that has been subjected to fractionation to remove various other components, and which composition substantially retains its expressed biological activity. Where the term “substantially purified” is used, this designation will refer to a specific binding agent composition in which the specific binding agent protein or peptide forms the major component of the composition, such as constituting about 50%, about 60%, about 70%, about 80%, about 90%, about 95% or more of the proteins in the composition.

Various methods for quantifying the degree of purification of the specific binding agent will be known to those of skill in the art in light of the present disclosure. These include, for example, determining the specific binding activity of an active fraction, or assessing the amount of specific binding agent polypeptides within a fraction by SDS/PAGE analysis. A preferred method for assessing the purity of a specific binding agent fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed specific binding agent protein or peptide exhibits a detectable binding activity.

Various techniques suitable for use in specific binding agent protein purification will be well known to those of skill in the art. These include, for example, precipitation with ammonium sulphate, PEG, antibodies (immunoprecipitation) and the like or by heat denaturation, followed by centrifugation; chromatography steps such as affinity chromatography (e.g., Protein-A or G-Sepharose), ion exchange, gel filtration, reverse phase, hydroxylapatite and affinity chromatography; isoelectric focusing; gel electrophoresis; and combinations of such and other techniques. It is believed that the order of conducting the various purification steps may be changed, or that certain steps may be omitted, and still result in a suitable method for the preparation of a substantially purified specific binding agent.

There is no general requirement that the specific binding agent always be provided in its most purified state. Indeed, it is contemplated that less substantially specific binding agent products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low-pressure chromatography system. Methods exhibiting a lower degree of relative purification may have advantages in total recovery of specific binding agent protein product, or in maintaining binding activity of an expressed specific binding agent protein.

It is known that the migration of a polypeptide can vary, sometimes significantly, with different conditions of SDS/PAGE [Capaldi et al., Biochem Biophys\Res Comm, 76: 425 (1977)]. It will therefore be appreciated that under differing electrophoresis conditions, the apparent molecular weights of purified or partially purified specific binding agent expression products may vary.

### Pharmaceutical Formulations

Generally, for pharmaceutical use, the compositions may be formulated as a pharmaceutical preparation comprising at least one specific binding agent with affinity for a pathogenic antigen conjugated to a radioisotope or radioisotope precursor, e.g., anti-gp120 antibody or mimetic conjugated to a chelating moiety that binds a radioisotope such as ⁶⁴Cu, and at least one pharmaceutically acceptable carrier, diluent or excipient and/or adjuvant, and optionally one or more further pharmaceutically active compositions.

Pharmaceutical compositions comprising antibodies are described in detail in, for example, U.S. Pat. No. 6,171,586. Such compositions comprise a therapeutically or prophylactically effective amount of a specific binding agent, such as an antibody, or a fragment, variant, derivative or fusion thereof as described herein, in admixture with a pharmaceutically acceptable agent. In a preferred embodiment, pharmaceutical compositions comprise specific binding agents that modulate partially or completely kill pathogenic particles or cells in admixture with a pharmaceutically acceptable agent. Typically, the specific binding agents will be sufficiently purified for administration to an animal.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the specific binding agent.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present disclosure, binding agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The pharmaceutical compositions can be selected for parenteral delivery. Alternatively, the compositions may be selected for inhalation or for enteral delivery such as orally, aurally, opthalmically, rectally, or vaginally. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this disclosure may be in the form of a pyrogen-free, parenterally acceptable aqueous solution comprising the desired specific binding agent in a pharmaceutically acceptable vehicle. A particularly suitable vehicle for parenteral injection is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

In another embodiment, a pharmaceutical composition may be formulated for inhalation. For example, a binding agent may be formulated as a dry powder for inhalation. Polypeptide or nucleic acid molecule inhalation solutions may also be formulated with a propellant for aerosol delivery. In yet another embodiment, solutions may be nebulized Pulmonary administration is further described in PCT Application No. PCT/US94/001875, which describes pulmonary delivery of chemically modified proteins.

It is also contemplated that certain formulations may be administered orally. In one embodiment of the present disclosure, binding agent molecules that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized Additional agents can be included to facilitate absorption of the binding agent molecule. Diluents, flavorings, low melting point waxes, vegetable oils, lubricants, suspending agents, tablet disintegrating agents, and binders may also be employed.

Pharmaceutical compositions for oral administration can also be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally also include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Another pharmaceutical composition may involve an effective quantity of binding agent in a mixture with non-toxic excipients that are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate [Sidman et al., Biopolymers, 22:547-556 (1983)], poly (2-hydroxyethyl-methacrylate) [Langer et al., J Biomed Mater Res, 15:167-277, (1981)] and [Langer et al., Chem Tech, 12:98-105 (1982)], ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc Natl Acad Sci (USA), 82:3688-3692 (1985); EP 36,676; EP 88,046; EP 143,949.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present disclosure is directed to kits for producing a single-dose administration unit. The kits may contain a binding agent optionally conjugated to a chelating agent or radioisotope, radio labeling reagents, prosthetic groups (bifunctional labeling reagents), an aqueous formulation and combinations thereof. Also included within the scope of this disclosure are kits containing single and multi-chambered pre-filled syringes (e.g., liquid syringes and lyosyringes).

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, a clinician may alter the dosage and modify the route of administration to obtain the optimal therapeutic effect. A typical dosage may range from about 0.01 mg/kg to up to about 100 mg/kg or more of a specific binding agent conjugated to a radioisotope, depending on the factors mentioned above. In other embodiments, the dosage may range from 0.01 mg/kg up to about 100 mg/kg; or 0.1 mg/kg up to about 100 mg/kg; or 1 mg/kg up to about 100 mg/kg.

For any, the therapeutically effective dose a binding agent conjugate can be estimated initially either in cell culture assays or in animal models such as mice, rats, rabbits, dogs, or pigs. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

### EXAMPLES

### Experimental Considerations

The SW infected rhesus macaque was employed as a model of pathogenic HIV infection. The viral gp120 was chosen as the in vivo target for detection and localization of SW infected cells and cell-free virions since it is accessible on the surface of both. One of the SIV Env specific monoclonal antibodies, that had previously been shown to bind to a wide range of cell free SW and SIV infected cells, was then selected to form the basis of the contrast agent. To maximize the sensitivity of imaging, PET was chosen as the imaging modality, and ⁶⁴Cu as the radionuclide because of its half-life of 12.7 hr, which is well suited for the labeling of long circulating ligands. In addition, ⁶⁴Cu is a high-energy positron emitter, which may be more sensitive for the generation of high-resolution images using PET, than gamma emitters such as ¹¹¹Indium or ⁹⁹Technetium, using SPECT. The isotope was chelated to the antibody with 1,4,7,10-tetraazacyclododecane-1,4,7,10-tetraacetic acid mono (DOTA NHS ester) because this chelator can be conjugated covalently to exposed lysines on the antibody. See Tamura et al. 64Cu-DOTA-trastuzumab PET imaging in patients with HER2-positive breast cancer. J Nucl Med. 2013; 54(11): 1869-75, and Mortimer et al. Functional imaging of human epidermal growth factor receptor 2-positive metastatic breast cancer using (64)Cu-DOTA-trastuzumab PET. J Nucl Med. 2014; 55(1): 23-9 and Guo et al., The role of p53 in combination radioimmunotherapy with 64Cu-DOTA-cetuximab and cisplatin in a mouse model of colorectal cancer. J Nucl Med. 2013; 54(9): 1621-9. Computed tomography (CT), was used to provide anatomical and signal localization information.

### ImmunoPET Probe

The SW Env specific monoclonal antibody (mAb) clone 7D3 was selected, based on its broad SIV Env specificity and the optimal signal to noise ratio noted from in vitro studies. This mAb was generated using SupT1 cells chronically infected with SIVmacCP-MAC, a lab-adapted variant of SIVmac251, and recognizes SIV Env on the surface of virus-producing cells. Edinger et al. found that 7D3 bound to the CCR5 binding site of gp120, and was effective at preventing syncytia formation in vitro with SIVmacCP-MAC, although it did not affect sCD4 binding, or neutralize SIVmac239. Furthermore, a more recent report demonstrated that three 7D3 antibody molecules can bind to the trimeric Env of SIVmacCP-mac and SIVmac239. See Edinger et al., Characterization and epitope mapping of neutralizing monoclonal antibodies produced by immunization with oligomeric simian immunodeficiency virus envelope protein. J Virol. 2000; 74(17): 7922-35 and White et al. Molecular architectures of trimeric SIV and HIV-1 envelope glycoproteins on intact viruses: strain-dependent variation in quaternary structure. PLoS Pathog. 2010; 6(12): e1001249

To mitigate the immunogenicity of xenogeneic mouse antibodies, decrease non-specific interactions, potentially improve the binding efficiency, and enable the chelation of ⁶⁴Cu, the mAb was simultaneously modified with linear 10 kD polyethylene glycol through succinimidyl ester-amino chemistry and DOTA NHS, via the same chemistry. See Wattendorf & Merkle, PEGylation as a tool for the biomedical engineering of surface modified microparticles. J Pharm Sci. 2008; 97(11): 4655-69 Transient expression of Env in Vero cells and SIVmac239-infected CEMx174 cells were used to verify binding of the conjugated 7D3 mAb by microscopy and flow cytometry. No detectable difference was noted between staining using modified and wild-type 7D3 mAb. Flow cytometry results demonstrated binding of 7D3 to the surface of infected CEMx174 cells under physiologic conditions.

### Characterization of Chronic SIV Infection

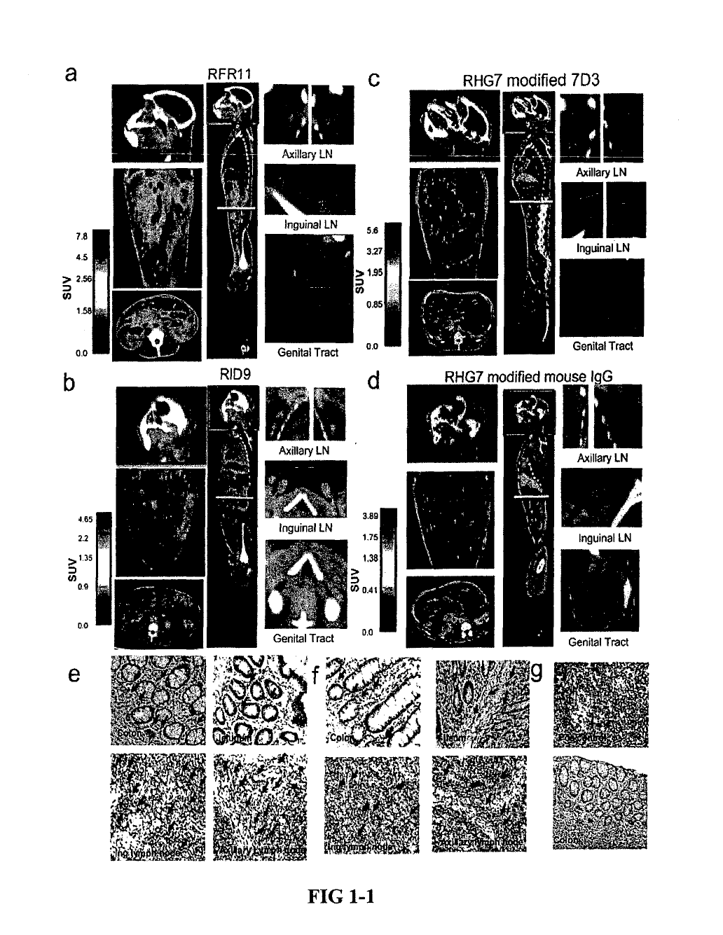

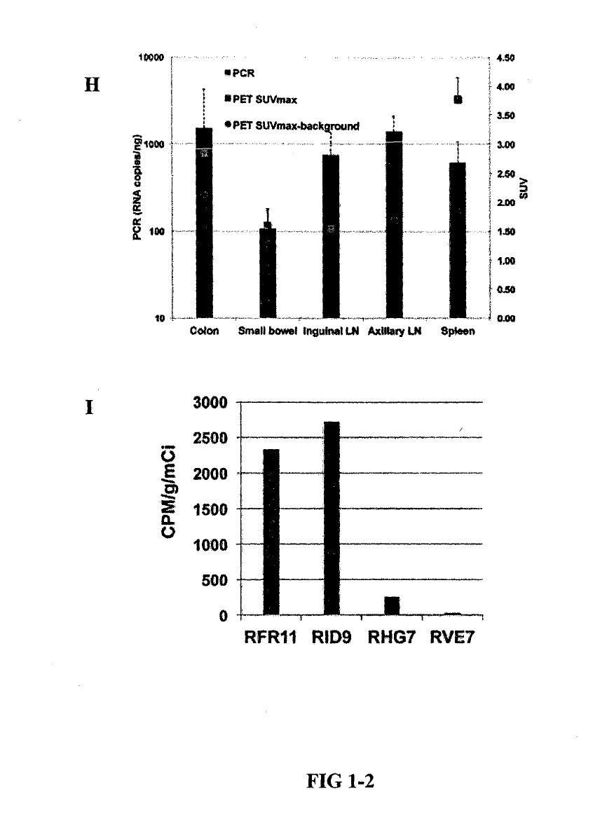

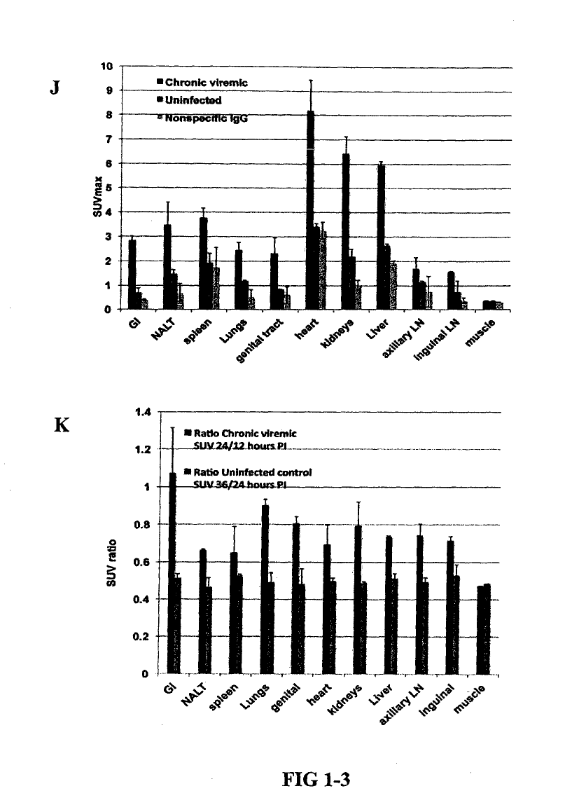

The procedure was then optimized for in vivo applications. Doses of 0.5-2.5 mg of this mAb were labeled with 1-3 mCi of ⁶⁴Cu chloride as described in the methods section. After removal of unbound ⁶⁴Cu, the probe was injected into rhesus macaques (4-15 kg) intravenously. PET/CT imaging was found to be optimal after a period of 24 to 36 hours post-injection, with improved antibody uptake in viremic animals when compared with uptake in control animals when both were imaged at shorter time points post-injection (data not shown). Macaques, chronically infected with SIVmac239, and non-infected control animals were imaged with the ⁶⁴Cu-labelled mAb 7D3 in order to test the ability of the modified mAb to specifically target and detect SIV infected cells and tissues. Fused frontal, sagittal, and axial, PET/CT images for chronically infected animals, RFR11 and RID9, injected with the probe are presented in FIG. 1a and 1b, while the images for the 7D3 probe and a similarly modified and labeled isotype control IgG are shown for the uninfected control macaque RHG-7 in FIGS. 1c and 1d.

In the viremic SIV infected animals, standard uptake value (SUV) maps were generated for each PET and corresponding CT image, with specific organs displayed in FIG. 1a-d. SUV values, greater than those observed in the control cases, were localized to the gastrointestinal (GI) system, specifically within the ileum, jejunum, and colon, and the axillary and inguinal lymph nodes. Of interest was also the detection of uptake within the lungs, a less well-characterized site of viral infection. In addition, antibody uptake was also detected consistently within the nasal cavity, likely reflective of the nasal associated lymphoid tissue (NALT) or nasal turbinates (FIG. 1a,b), an area that has received little attention as a source of viral replication. In the males, uptake was frequently observed within the genital tract, specifically in the vas deferens and epididymis. Infection of epithelial cells was not detected in the male genital organs. In the uninfected monkeys, background was evident within the liver, heart, kidneys and spleen, which is typical of antibody-based contrast agents.

Quantification of PET Imaging and Comparison with qRT-PCR and Immunohistochemistry (IHC)

The imaging signal data was verified by either qRT-PCR and through the examination of sections of the specific tissue of interest using IHC for gag (FIG. 1e-h). These confirmatory studies utilized either rectal biopsy tissues obtained immediately post imaging and/or tissues obtained post-mortem. From the IHC data it is clear that the gastrointestinal tract, lymph nodes and spleen, all contained infiltrating SW infected cells of lymphocyte or macrophage morphology. Negative control tissue (FIG. 1g) did not contain any detectable signal using the same IHC protocol. qRT-PCR was also performed, using tissue samples from the colon, small bowel (jejunum and ileum), inguinal/axillary lymph nodes, and the spleen for all of the chronically infected animals and controls (FIG. 1h). In all cases, corroborating our IHC results, viral RNA was detected, with the highest levels in the colon; no RNA was detected in the controls. In addition, the ⁶⁴Cu-radioactivity associated with aliquots of rectal biopsies from viremic monkeys RFR11, RID9 and uninfected controls RHG7, RVE7 was quantified with a scintillation counter. The signal from these tissues, normalized for the mass of the biopsy and the total amount of radioactivity administered was 17.6 times higher in infected animals (FIG. 1i), versus both controls, providing additional confirmation of the specificity of the PET imaging.

To compare the PET results from viremic animals to those of the controls, the data was quantified using standard uptake values (SUV) (see FIG. 1j). Volumes of interest were chosen using the PET/CT fusion images; the CT images identified the organ and an outline of the organ was made manually. The outline was then extended in depth until the organ volume was defined. Using the volume as the region of interest (ROI), the maximum SUV within that organ was then determined Once this was completed, the SUVmax within viremic and uninfected animals, was compared within the same graph. along with qRT-PCR results from the corresponding colon, small bowel, inguinal/axillary lymph nodes and the spleen of the same animal (FIG. 1h). The PET SUVmax values mimic the general trends of the PCR data. For the spleen, which tends to have higher background uptake, the SUVmax minus background is a more relevant comparison with the qRT-PCR data.

Next, the SUVmax measurements for various tissues from chronically infected and uninfected macaques, injected with 7D3 labeled antibody, and from uninfected macaques, injected with a labeled isotype control antibody, were compared (FIG. 1j). From the results shown in FIG. 1j, the average SUVmax, measured 24 hours post injection in viremic animals was 2.84 (GI tract), 3.47 (nasal cavity, NALT), 3.77 (spleen), 2.44 (lungs), 2.3 (genital tract), 1.68 and 1.54 (axillar and inguinal lymph nodes), and 0.35 (muscle). Within the uninfected controls, the average SUVmax was measured to be 0.7 (GI tract), 1.49 (NALT), 1.95 (spleen), 1.18 (lungs), 0.85 (genital tract), 1.1 and 0.72 (axillar and inguinal lymph nodes) and 0.362 (muscle). There is increased uptake within organ systems likely to contain virus or virally infected cells and tissue.

A global comparison of the PET measurements for each animal was then performed that included the signals of all organs. This was achieved applying a fully nested hierarchical ANOVA model for the SUVmax response. When the viremic animals were compared with uninfected controls, the p-value was 7.39E-6, indicating that the imaging data was statistically significant. Next the animals within each group, infected or uninfected, were compared for homogeneity, and the p-value was 0.89, indicating that the individual animals within each group were not significantly different from each other. The analysis also showed that both the infection status and organs contributed significantly to the SUVmax value, and that for chronically viremic animals, and both control groups, the measurements for each organ within each group, were significantly different from each other, yielding p-values of 1.4E-09, 7.1E-27 and 4.78E-19 respectively. In addition, when the Kruskal-Wallis test was applied for each organ separately, the organ specific differences from each animal showed that for each organ measured, except muscle, the signals measured in viremic monkeys and uninfected controls were statistically distinct.

Another method of determining whether uptake was specific for a particular organ is to examine the dynamics of uptake, through the acquisition of sequential images at multiple time points post-injection. In our case, due to logistics, scanning was performed at 12, 24 and 36 hours post injection in select cases. In FIG. 1k ratios of the average SUVmax values at each time point were plotted for both cases and for each organ system. When the viremic monkeys were compared with the aviremic controls, all of the ratios were higher, typically above 0.6, with the GI tract giving values >1.0, indicating continued specific uptake of the probe. Uptake was not detected in the central nervous system (CNS) in either SIV infected monkeys or uninfected controls.

### SIV Localization Before and During Antiretroviral Therapy (ART)

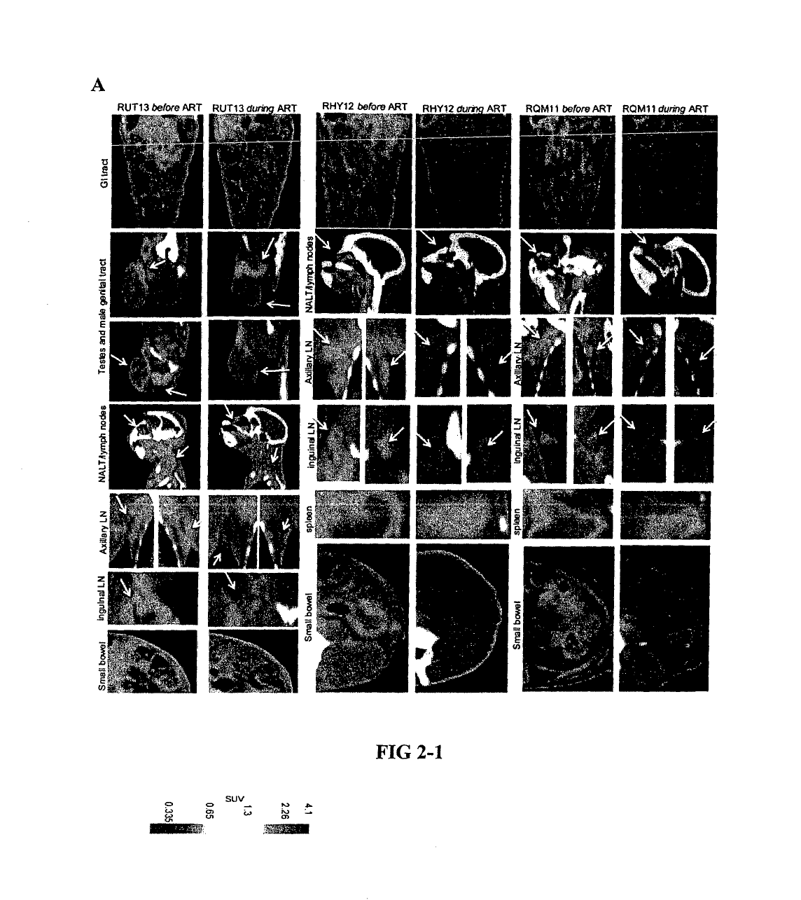

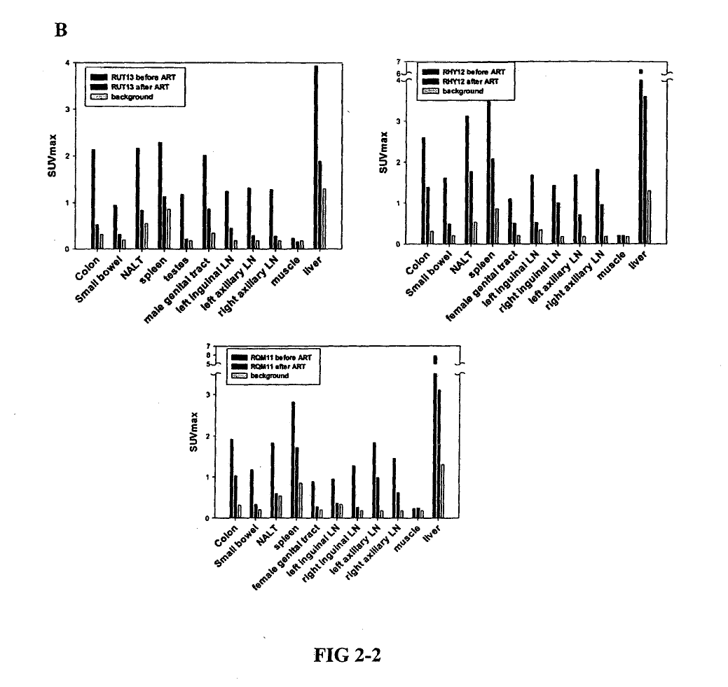

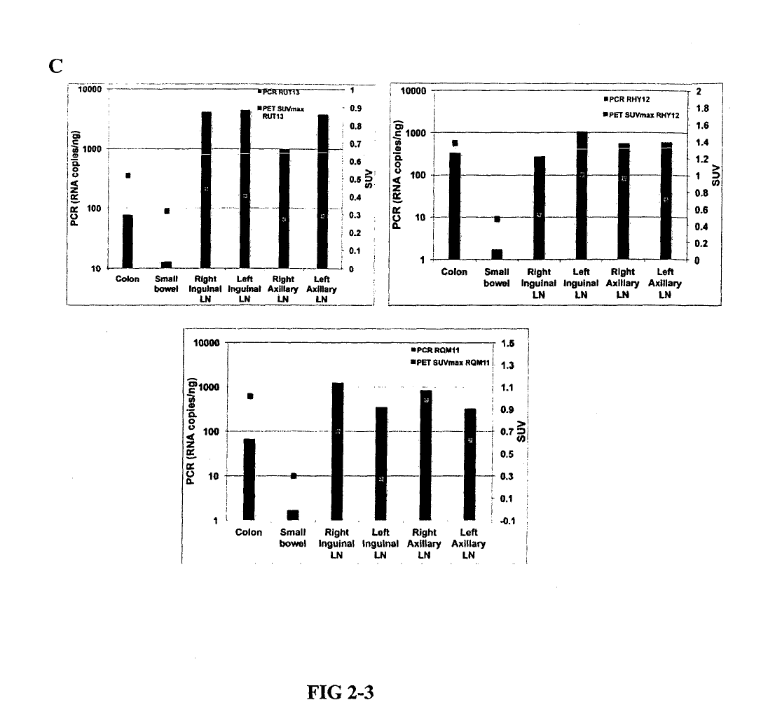

In order to both confirm that this methodology was sufficiently sensitive to study SIV infections and locate reservoirs, three chronically infected animals (RUT13, RHY12, and RQM11) were first imaged 36 hours post injection with our modified 7D3 agent, FIG. 2, and then initiated on ART (20 mg/kg/d PMPA and 50 mg/kg/d FTC courtesy of Gilead Inc each subcutaneously and 100 mg/d×40 days of L′870812 courtesy of Roche). All three animals were aviremic by 3-4 weeks of treatment, (viral loads <60 copies RNA/ml, the sensitivity of the assay). They were then imaged again at 5 weeks post ART (FIG. 2). In FIG. 2a, single plane images of the overall GI tract, nasal cavity, axillary/inguinal lymph nodes, spleen and small bowel are presented for each animal before and on day 34 of treatment. From these images, there was measurable SIV signal localized within the GI tract, NALT, genital tract, axillary and inguinal lymphoid tissue, prior to treatment, similar to FIG. 1. After 34 days of treatment, all organ systems exhibited decreased uptake (FIG. 2a,b). However, there was residual signal (above the background measured in control animals) in all organ systems, with moderate SUVmax values still remaining in the colon, spleen, male genital tract, NALT, and individual lymph nodes for specific animals. In all cases, the SUVmax did not decrease to our measurable limit (background). To assess the statistical significance of the SUVmax measurements, the same hierarchical ANOVA analysis was performed as above. In this case, the same animals were compared before and after five weeks of ART, using the SUVmax data from all of the organs imaged. In this case, the differences between both conditions were significant, with a p-value of zero. The model, via a pairwise comparison also showed that RUT13 and RQM11 were significantly different from RHY12, with a p-value of 0.0027, demonstrating the individual variation in ART treatment.

To verify our imaging results, qRT-PCR was performed on multiple tissue samples that included colon, small bowel, right and left inguinal/axillary lymph nodes and spleen collected at necropsy performed on days 39/40 post ART (FIG. 2c). The maximum RT-PCR values were compared directly with the SUVmax data in FIG. 2c, with both values plotted on the same graph. From FIG. 2c, even though PET measures env protein and qRT-PCR viral RNA for virus localization, there was indeed residual virus or infected cells in the locations identified by PET. It should be noted that both the spatial variation within an animal and the variation between animals suggested by PET, was confirmed with qRT-PCR data, with two orders of magnitude variation within an organ and between animals. In addition, the nasal turbinates, genital tract, and lung samples were all positive, indicating virus localization during both chronic and treated conditions, similar to what was observed in viremic animals.

### SIV Localization in Elite Controllers

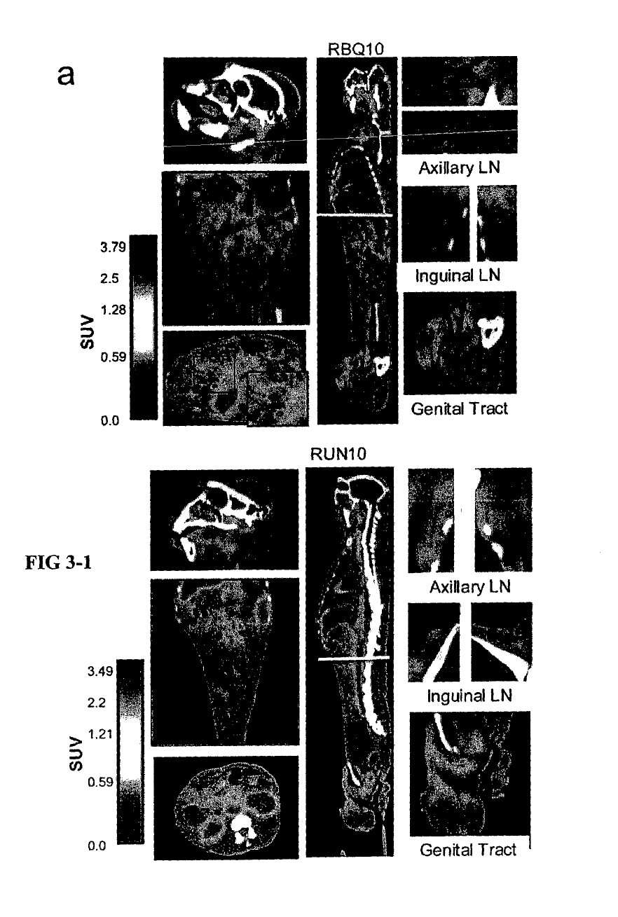

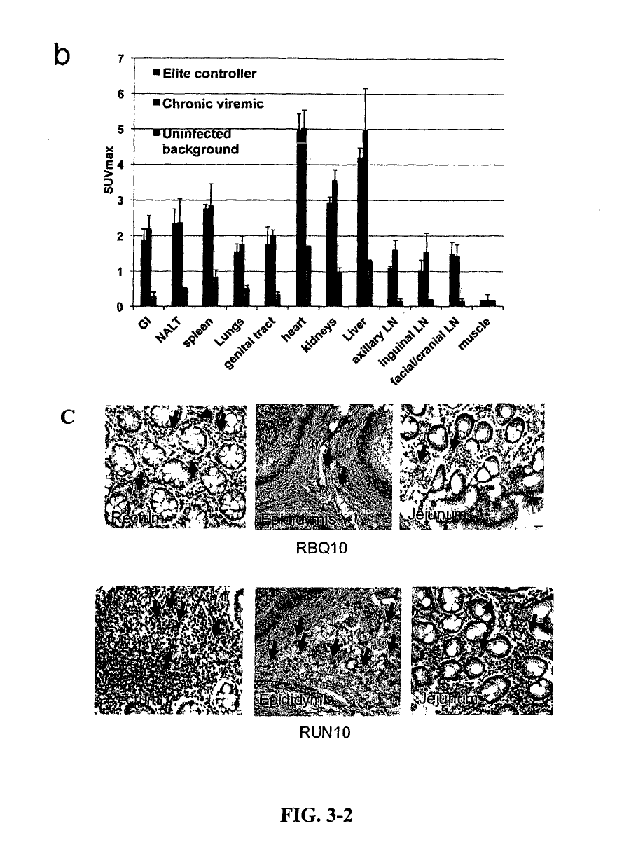

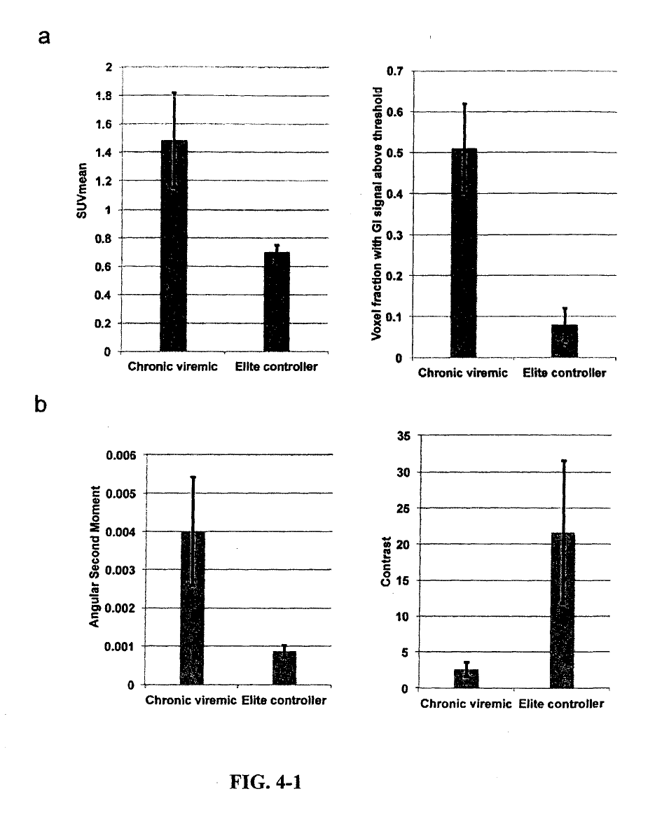

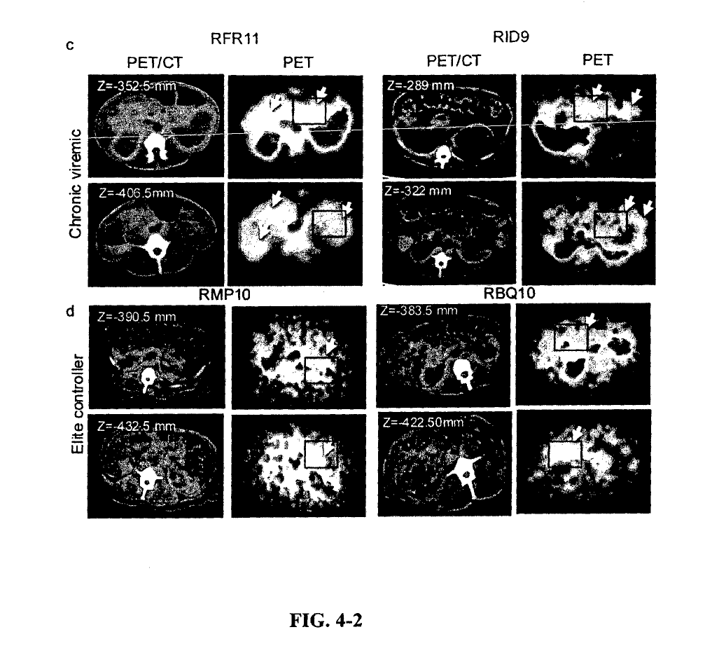

The methodology was then applied to SIV infected elite controllers (EC). ECs are individuals that naturally suppress SW (or HIV) replication to undetectable levels in plasma for extended periods of time without antiretroviral intervention. The study of viral persistence in these individuals is challenging. EC monkeys exhibited detectable uptake (FIG. 3a) within the GI tract, genital tract, NALT, lungs, spleen and axillary lymph nodes. These imaging data were supported by IHC in biopsy samples from the rectum, epididymis, and jejunum. The uptake though, was restricted to specific small regions or foci in the ECs. In FIG. 3b the organ signal quantified with SUVmax surprisingly appeared to approximate the results of the viremic animals. However, when the hierarchical ANOVA was applied, it was found that overall, the PET SUVmax data for the viremic animals was statistically distinct from the ECs. In order to clarify the differences between the ECs and viremic animals, the SUVmean was measured within the GI tract (FIG. 4a). In addition, the voxel fractions (fraction of total volume of GI tract) were compared (FIG. 4a). From the SUVmean measurement and voxel fraction data, it was found that the GI tract gave values that were 2.1 and 6.38 times greater, respectively, in the viremic as compared with the EC animals. While the viremic macaques and ECs contain regions of comparably high uptake, in the ECs, this was spatially restricted to much smaller volumes of tissue and thus the overall probe uptake was lower. Additional metrics quantifying the spatial distributions within the GI tract were calculated (FIG. 4b,c), further supporting this conclusion.

### ⁶⁴Cu Antibody Labeling

Two monoclonal antibodies and hybridomas, designated 7D3 and 36D5, were acquired and tested in vitro using Vero cells, transiently transfected with the pSRSEB vector and CEMx174 cells infected with SIVmac239. See Edinger et al., J Virol, 2000, 74(17): 7922-35. The monoclonal antibody secreting hybridoma (clone 7D3) was obtained from Dr. James Hoxie at the University of Pennsylvania. Both 7D3 and 36D5 MAbs are non-overlapping (binding a conformational epitope in the CCR5 binding site and V3 respectively). These two antibodies were chosen based on their ability to bind infected cells using flow cytometry and because 7D3 was shown to be non-neutralizing, while 36D5 is considered able to block binding to CCR5. The pSRSEB vector expresses the wild-type env protein (gp160) of SIVmac239 and blue fluorescent protein (BFP) and supports high levels of expression. SIVmac239 was chosen because experimental infection of rhesus monkeys with cloned SIVmac239 have exhibited the most consistent behavior, i.e., 50% of the infected macaques died within 1 year with characteristic SIV-induced immunodeficiency disease.

Antibodies are DOTA and PEG labeled prior to usage and lyophilized for storage. See Li et al., J Nucl Med, 2010, 51(7):1139-46. Due to difficulties cleaving the IgG1 mouse monoclonals, polyethylene glycol (mPEGNHS ester) (10kD) was conjugated to the antibodies to decrease their immunogenicity. The DOTA-NHS ester chelates with the ⁶⁴Cu. Care was taken to test the DOTA and PEG conjugated antibodies in vitro to make sure they would still bind to gp120-optimized using the following protocol:

1. Dilute 500 μg of MAb/PEG/DOTA conjugate in 100 μl of 0.1 M NH4OAc, pH 5.5

2. Dilute ⁶⁴CuCl₂ with same buffer such that final concentration 250 μCi/μl. Example: 30 mCi would arrive in 5-7 μl, 6 mCi/μL; add 115 μL of 0.1 M NH4OAc, pH 5.5, to make a concentration of 250 μCi/μl.

3. Added 20 μl of the diluted ⁶⁴CuCl₂ to the MAb/PEG/DOTA conjugate, creating a 120 μl solution, and incubated this at 37° C. for 1 hour.

4. Samples of this solution were then taken for thin layer chromatography (TLC), and then the solution was quenched with 5M EDTA for 5 minutes. Again a sample of the quenched compound was taken for comparison using TLC.

5. If the TLC yielded >75% uptake of ⁶⁴Cu then was purified in a size exclusion column within the hot cell and resuspended in sterile saline for administration to the animals. This provided one dose typically at ˜2 mCi.

### Antibody Modification for In Vivo Imaging

To conjugate 1,4,7,10-tetraazacyclododecane-1,4,7,10-tetraacetic acid mono (DOTA NHS) and polyethylene glycol esters (PEG ester) to surface lysine residues of 7D3, molar ratios of 60:1 and 20:1 respectively were used. Briefly, 1 mg of clone 7D3 mAb (at 3.8 mg/mL) was buffer exchanged with 0.1M phosphate buffer (EMS) pH 7.3 plus chelex 100 (Biorad) using a 10 kDa Amicon spin column (Millipore). Then 1 uL of 0.5M DOTA NHS-ester (Macrocyclics) in phosphate buffer and chelex 100 and 32 μl of 5 mM m-PEG-SMB 10K (Nectar), also in phosphate buffer and chelex 100, were added and reacted for 4 hrs at RT on a rotator. Unconjugated reagents were removed using 30KDa Amicon spin columns in phosphate buffer plus chelex 100. Samples were quantified via UV-VIS spectroscopy, and the conjugations verified via gel electrophoresis using Tris acetate gels in TA running buffer with SDS (Invitrogen). The modified 7D3 antibody was then aliquoted and lyophilized for storage.

Animals were imaged using a Siemens Biograph 40 PET/CT, using image settings for 64Cu. Typically between 250 and 300 slices were compiled for each macaque depending on body size.

SIV Infection of 174xCEM Cells and Flow Cytometry

The human TxB hybridoma 174xCEM cell line was obtained through the NIH AIDS Reagent Program, Division of AIDS, NIAID, NIH (donated by Dr. Peter Cresswell). Ten million SIV susceptible 174xCEM cells were suspended in 1 ml of RPMI 1640, with 10% fetal bovine serum, and inoculated with 20 μl of SIVmac239 stock passaged in 174xCEM cells (TCID50 ˜1×10⁵/ml). The cells were incubated at 37° C. in a 5% CO₂ incubator for 1 hour then diluted to a volume of 20 ml. Media was replenished every 3 days maintaining the concentration at 1×10⁶ cells/ml. On day 10 post-infection, cells were harvested for staining and an aliquot analyzed by flow cytometry with anti-SIV 7D3 conjugated to dylight 649 (Thermo Science,). Aliquots of 500,000 cells were suspended in 200 μl of PBS containing different amounts of 7D3 (1000, 250, 63, 4, 1 and 0.25 ng). Cells were incubated for 15 minutes at room temperature, washed in 2 ml of PBS with 2% FBS and fixed in 200 μl of fresh 1% paraformaldehyde in PBS. Uninfected cells were similarly stained. Flow cytometry was performed using a LSRII (BD BioSciences) and the data obtained analyzed using FlowJo software (version 9.2; TreeStar).

### Immunofluorescence

The ability of the PEG/DOTA/7D3 to bind gp120 was tested via immunofluorescence in both SIV Env (pSRSEB plasmid) transfected or control Vero cells, as well as SIV infected or control 174xCEM cells. Vero cells were transfected using Lipofectamine 2000 (Invitrogen) according to the manufacturer's protocol; 48 h post transfection cells were fixed and permeabilized in 50% methanol:acetone for 10 minutes at −20° C., blocked with 5% BSA and stained using 1.8 ng/μl PEG/DOTA/7D3 and an Alexafluor488 donkey-anti-mouse (Invitrogen) antibody. In addition, non-infected and day 10 SIV infected 174xCEM cells, cytospun onto glass slides, were fixed in 50:50 methanol/acetone. After fixation, cells were blocked with 5% BSA and stained using as primary antibody 2 ng/μl PEG/DOTA/7D3 or unmodified 7D3 and developed using an Alexafluor488 donkey-anti-mouse antibody.

### Radiolabeling

Lyophilized PEG/DOTA/7D3 was re-suspended in chelexed 0.1M NH4OAc pH 5.5 (Sigma). Copper (II)-64 chloride (Washington University, MO) was diluted similarly; they were then mixed together at a ratio of approximately 5 mCi/mg and incubated at 37° C. for 1 hour. The antibody conjugates typically labeled in the range of 1 mCi/mg-3.5 mCi/mg per dose. Each dose was buffer exchanged with pharmaceutical grade saline 3 times using a 10kD centrifugal filter to a final volume of 20 μl. The conjugated mAb was then added to 1 ml of pharmaceutical grade sterile saline in a sterile glass vial. Uptake was confirmed on an aliquot using thin layer chromatography. The labeled antibody conjugates gave values in the range of 1 mCi/mg-3.5 mCi/mg

### Chimeric MAb Based on the Variable Regions of the 7D3 and 36D5 Mouse MAb

One creates chimeric MAbs with the variable regions of the 7D3 and 36D5 fused to the rhesus macaque IgG molecules. This molecule could be custom made for optimal tissue penetration and shorter circulation half-life than a traditional MAb. Techniques for producing “primatized antibodies” and primate recombinant cytokine-IgG fusion constructs are known. See Klatt et al., J Clin Invest, 2008, 118(6):2039-49. Typically constructs use a macaque IgG2 backbone with mutated FcR binding sites and complement binding sites to prevent the activation of complement of cell mediated lysis. One creates chimeric antibodies with the mouse Ig domain containing the CDRs fused to this macaque IgG2 heavy and either kappa or lambda chains to create a complete IgG. The full length and Fab fragments will be tested in vitro for binding and staining intensity into infected tissues using in situ histochemistry. Primatized MAbs use a human Ig domain in which the murine CDRs are transplanted, linked to primate heavy and light chains. Based on the binding data observed with the macaque-mouse chimeric MAbs, one selects a IgG backbone to transplant the murine CDRs into the complete macaque IgG molecule, using DNA synthesis for the CDR containing Ig domain. This will result in a macaque IgG with only mouse CDRs. Catalent is a commercial company which uses a lentiviral protein production system to mass produce MAbs.

Fully Human Monoclonal Antibodies (mAbs) to Human Gp120

One generates fully human mAbs from nonhuman variable regions using information from the human germline repertoire. Residues within and proximal to CDRs and the V_{H}/V_{L} interface of 7D3 and 36D5 are iteratively explored for substitutions to the closest human germline sequences using semi-automated computational methods. See Bernett et al., J Mol. Biology, 2010, 396(5):1474-1490, hereby incorporated by reference in its entirety. One generates fully human antibodies with substitutions compared to the parent murine sequences. Substitutions may be in the CDRs.

The engineering process to generate fully human mAbs from murine Fvs consists of five main steps: (1) design of framework-optimized V_{H} and V_{L} template sequences of 7D3 and 36D5 (2) identification of the closest matching human germline sequence for the framework-optimized VH and VL, (3) screening of all possible single substitutions that increase the sequence identity of the framework-optimized sequence to the closest human germline sequence, (4) screening of V_{H} and V_{L} variants consisting of combinations of neutral or affinity enhancing single substitutions, and (5) screening of the highest-affinity V_{H} and V_{L} pairs to generate the final fully human mAb.

One defines two principal scores used to measure sequence humanness. Human identity is defined as the number of exact sequence matches between the Fv and the highest identity human germline VH, Vκ, JH, and Jκ chains (the D-segment for the heavy chain is not included). The second score is the number of total “human 9-mers”, which is an exact count of 9-mer stretches in the Fv that perfectly match any one of the corresponding stretches of nine amino acids in our set of functional human germline sequences. Both human 9-mers and human identity are expressed as percentages throughout in order to enable comparison between antibody Fvs of different lengths.

1. A binding agent specific for pathogenic antigen, wherein the binding agent is conjugated to a molecule with a positron-emitting radionuclide.

2. A binding agent of claim 1, wherein the pathogenic antigen is a virus particle surface antigen.

3. The binding agent of claim 1 which is an antibody, antibody fragment, aptamer, or antibody mimetic.

4. The binding agent of claim 3, wherein the antibody epitope is on a gp120 protein of a lentivirus.

5. The binding agent of claim 3, wherein the antibody epitope is on the V3 loop of a gp120 protein of a lentivirus.

6. The binding agent of claim 3, wherein the antibody is a humanized antibody or human chimera.

7. The binding agent of claim 6, wherein the human chimera comprises a polypeptide sequence selected from
a) a variable domain of the light chain from an antibody conjugated to a human immunoglobulin;
b) a variable domain of the heavy chain from an antibody conjugated to a human immunoglobulin; or
c) a variable domain of the light chain and heavy chain from an antibody conjugated to a human immunoglobulin.

8. The binding agent of claim 6, wherein the humanized antibody comprises polypeptide sequences of complementarity determining region one (CDR-1), CDR-2, and CDR-3 on the light (VL) chain of an antibody and polypeptide sequences of CDR-1, CDR-2, and CDR-3 heavy (VH) chains of an antibody.

9. The binding agent of claims 6-8, wherein the antibody is selected from CD4BS, CH103, PG V04, PGT-127, PGT-128, PGT-130, PGT-131, CH01, CH02, CH03, and CH04, 2909, VRC01, VRC02, VRC03, HJ16, HGN194, HK20, PG9, PG16, 22A, 171C2, 71B7, 36D5, 31C7, 8H1, 189D5, 77D6, 3E9, 4B11, 5B11, 7D3, 8C7, 11F2, 17A11, 2G12, b12, b13, m18, F105, and 447-52D.

10. The specific binding agent of claim 1, wherein the positron-emitting radionuclide is ⁷¹As, ⁷²As, ⁷⁴As, ⁷⁶Br, ¹¹C, ^{34m}Cl, ⁵⁵Co, ⁶²Cu, ⁶⁴Cu, ¹⁸F, ⁵²Fe, ⁶⁶Ga, ⁶⁸Ga, ¹²⁴I, ⁵²Mn, ¹³N, ¹⁵O, ⁸²Rb, ^{94m}Tc, ⁸⁶Y, and ⁸⁹Zr.

11. The binding agent of claim 1, wherein the molecule comprises 1,4,7,10-tetraazacyclododecane.

12. The binding agent of claim 3, wherein the antibody is conjugated to a hydrophilic polymer.

13. The binding agent of claim 12, wherein the hydrophilic polymer is polyethylene glycol.

14. The binding agent of claim 4, wherein the lentivirus is a simian immunodeficiency virus or human immunodeficiency virus.

15. A method of imaging a lentiviral infection comprising,
a) administering a tracer composition comprising a specific binding agent of claim 1 to a subject;
b) detecting pairs of gamma rays emitted by the positron-emitting radionuclide; and
c) generating an image indicating a location of the positron-emitting radionuclide within an area of the subject.

16. A method of treating or preventing a lentiviral infection comprising administering an effective amount of a specific binding agent for a lentivirus envelope protein, wherein the binding agent is conjugated to a molecule with a radioisotope to a subject in need thereof.

17. The method of claim 16, wherein the radioisotope is selected from ¹¹¹In, ¹³¹I, ⁹⁰Y, ¹⁷⁷Lu, ¹⁸⁶Re, ¹⁸⁸Re, ⁶⁷cu, ²¹¹At, ²¹²Bi, ²¹³Bi, ²²⁵Ac, ¹²⁵I, and ⁶⁷Ga.

18. The method of claim 16, wherein the subject is human.

19. The method of claim 16, wherein the specific binding agent is administered in combination with another antiviral agent.

20. The method of claim 19, wherein the antiviral agent is selected from abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, elvitegravir famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, MK-2048, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate (TAF), tipranavir, trifluridine, trizivir, tromantadine, truvada, valaciclovir, valganciclovir, vicriviroc, vidarabine, viramidine, zalcitabine, zanamivir, or zidovudine, and combinations thereof.
